(19) United States
(12) Patent Application Publication
(10) Pub. No.: US 2021/0315474 A1
(43) Pub. Date: Oct. 14, 2021

(54) DECOUPLED COIL ASSEMBLIES, MAGNETIC RESONANCE SYSTEMS AND METHODS OF USE

(71) Applicant: FluoID Inc, Magog (CA)

(72) Inventors: Chathura Kumaragamage, East Haven, CT (US); Axel Patrick Mathieu, Magog (CA)

(21) Appl. No.: 17/271,796

(22) PCT Filed: Aug. 30, 2019
(86) PCT No.: PCT/CA2019/051221

§ 371 Date: Feb. 26, 2021

Related U.S. Application Data
(60) Provisional application No. 62/724,997, filed on Aug. 30, 2018.

(51) Int. Cl. A61B 5/055; G01R 33/3415; G01R 33/30; G01R 33/36

(52) U.S. Cl. CPC A61B 5/055; G01R 33/3415; G01R 33/3635; G01R 33/3657; G01R 33/30

(57) ABSTRACT

Provided are coil assemblies, and holding assemblies, devices and systems comprising the coil assembly as well as methods of use thereof. The coil assembly includes a first radio frequency coil element configured for transmitting a first radio frequency signal through a region of interest of a subject or test sample, the first radio frequency signal for exciting a first spin species in the region of interest. The coil assembly also includes a second radio frequency coil element configured for resonating at a second radio frequency signal to receive a magnetic resonance signal from a second spin species from the region of interest. The first radio frequency signal and the second radio frequency signal are separated by a frequency interval. A drive circuitry is connected to the second radio frequency coil element. A first decoupling circuit prevents coil coupling between the first radio frequency coil element and/or a transmitter coil element. A second decoupling circuit prevents coil coupling between the second radio frequency coil element and the transmitter coil element.

This is a Patent Cooperation Treaty Application which claims the benefit of 35 U.S.C. § 119 based on the priority of U.S. Provisional Patent Application No. 62/724,997 filed Aug. 30, 2018, which is incorporated by reference in its entirety.

### FIELD OF THE DISCLOSURE

The present disclosure relates to the field of nuclear magnetic resonance, such as magnetic resonance imaging (MRI) and magnetic resonance spectroscopy (MRS), in particular systems and methods for MRI and MRS. The present disclosure relates as well to a coil assembly for use in a MRI/MRS system.

### BACKGROUND OF THE DISCLOSURE

Harnessing the power of the physical phenomenon of nuclear magnetic resonance (NMR) has had far-reaching impact in research, engineering and medicine. For example, MRS has enabled comprehensive studies on the solution and solid-state structures of organic and inorganic substances, as well as insight into their electronic, chemical and physical characteristics. Similarly, MRI has become an indispensable medical diagnostic tool that, importantly, does not require the use of ionizing radiation. It has been widely exploited to provide detailed information on the anatomy of organisms, including man, and for the study of physiological processes occurring in biological systems.

In order to take full advantage of NMR for these wide-ranging applications, systems can have the appropriate capabilities to first generate NMR in a test sample, whether it is a chemical compound or the human body, then accurately detect, record and process the data from that effect. For this, systems have been developed with a variety of configurations and components with some available commercially. However, due to the large number of NMR detectable spin species and the variety of potential applications, different, sometimes specialized, components of these systems are necessary.

Magnetic resonance (MR) is a characteristic of particular atomic isotopes containing an odd number of protons and/or neutrons, since such nuclides have an intrinsic magnetic moment and angular momentum (i.e. a non-zero spin). In contrast, those with even numbers of both atomic particles have a zero spin. When placed in a strong, uniform and static magnetic field (B0), the magnetic moment of the isotopes with non-zero spins, or spin species, present in a test sample become aligned, or polarized, either with or against the direction of B0, and produce a net magnetization in the same. However, a perturbation from this equilibrium state (rotation of net magnetic moment into transverse plane) is required in order to produce information about the test sample. This is typically achieved by causing the nuclear magnetic moments to mutate their alignment away from B0 through exposure to a second radio frequency (RF) magnetic field (B1) at a specific frequency corresponding to one particular type of nuclei (Larmor frequency).

When B1 is removed, the nuclei relax back toward their equilibrium state (termed relaxation), producing a time-varying magnetic field that can be detected (MRI signal). Since the detectable signal is at a specific resonance frequency that depends on the strength of the magnetic field, as well as the particular magnetic properties of the isotope, MR permits the observation of detailed properties of the atomic nucleus and the characteristics of the molecular environments in which the nuclei reside. These signals are detected, measured, then processed to reconstruct the data into an image representation, as in MRI, or derive spectral information, as in MRS, for the nuclei concerned. One of the key characteristics of MR is that the resonance frequency of a particular substance is directly proportional to the strength of the applied magnetic field.

One of the key components of the MR system is the radio frequency coil (RF) element. This is employed for transmission of RF signals to perturb the spin species and receiving the RF signals produced upon relaxation of the spin species. The same or a different RF coil element may also be used for generating and applying the B1 field to the test sample at a defined frequency. Due to their intimate involvement with the magnetic resonance process, significant attention has been devoted to designing and developing these components of the MR system, as well as developing ways for using multiple RF coil elements within the same system. However, RF coil elements that are set to the same or nearby radiofrequencies cannot easily be positioned in close proximity since they may couple to each other, adversely affecting signal quality. Such coupling also can cause overheating of the coil elements and may even result in their physical destruction during power intensive applications.

WO2008/152511A1 is directed to a dual nuclear MR transition line resonator and operating at X pairs, with X being ³¹P, ²³Na, ³He, or ¹²⁹Xe.

### SUMMARY OF THE DISCLOSURE

The present disclosure provides coil assemblies and systems for acquiring magnetic resonance (MR) data. In particular, these systems can be employed in magnetic resonance imaging (MRI) and magnetic resonance spectroscopy (MRS). As such, they have utility in a number of therapeutic, diagnostic, or research applications.

According to one aspect, there is disclosed a coil assembly including:

a first radio frequency coil element configured for transmitting a first radio frequency signal through a region of interest of a subject or test sample, said first radio frequency signal for exciting a first spin species in the region of interest, and

a second radio frequency coil element configured for resonating at a second radio frequency signal to receive a magnetic resonance signal from a second spin species from the region of interest,
the first radio frequency signal and the second radio frequency signal being separated by a frequency interval;
corresponding circuitry connected to the first and second radio frequency coil elements;
a first decoupling circuit configured for preventing coil coupling between the first radio frequency coil, the second radio frequency coil element and/or a second transmitter coil element, the second transmitter coil element optionally being external to the coil assembly, the decoupling circuit comprising:
   a junction at each end wherein each junction is connected to the first radio frequency coil element, the first decoupling circuit is tuned to the second radio frequency signal, and
   a separation distance between the junctions of the first decoupling circuit is configured for reducing the electric field caused by the proximity between the first and second radio frequency signals; and

a second decoupling circuit configured for preventing coil coupling between the second radio frequency coil element, the first radio frequency coil element and/or the second transmitter coil element, the second transmitter coil element configured to transmit the second radio frequency signal for exciting the second spin species in the region of interest,
the second decoupling circuit being connected to the second radio frequency coil element, the second decoupling circuit configured to disable the second radio frequency coil element when the transmitter coil element operating at the second frequency is active, and/or when the first radio frequency coil element operating at the first radio frequency is active, and

a power means for powering the second decoupling circuit.

In some embodiments, the second decoupling circuit comprises a switch.

For example, the first decoupling circuit is configured for preventing coil coupling between the first radio frequency coil element and the second radio frequency coil element.

For example, the first decoupling circuit is configured for preventing coil coupling between the first radio frequency coil element and the transmitter coil element.

For example, the first decoupling circuit is configured for preventing coil coupling between the first radio frequency coil element and the second radio frequency coil element and between the first radio frequency coil element and the transmitter coil element.

For example, the second decoupling circuit is configured for preventing coil coupling between the second radio frequency coil element and the first radio frequency coil element.

For example, the second decoupling circuit is configured for preventing coil coupling between the second radio frequency coil element and the transmitter coil element.

For example, the second decoupling circuit is configured for preventing coil coupling between the second radio frequency coil element and the first radio frequency coil element and between the second radio frequency coil element and the transmitter coil element.

The region of interest of a subject or test sample, can be the entire subject or test sample or a part thereof such as a body part, organ, or portion of a material. The test sample can for example comprise cells for example growing on a tissue culture plate or in a 3D culture system, or printed using a 3D printing system.

For example, the first spin species is different from the second spin species.

For example, a minimum separation distance for a first decoupling element can be calculated based on at least the transmit power and electric field on the first radio frequency coil element.

For example, a minimum separation distance for a first decoupling element can be determined according to the package size of fixed valued capacitors that are compatible to be used at the operated peak RF power.

The first decoupling circuit can be a passively decoupled circuit. For example, the first decoupling circuit includes at least one capacitive element; and, an inductive element, which can be in parallel with the capacitive element.

For example, the second decoupling circuit is configured to inhibit the second radio frequency coil element from resonating when the transmitter coil element is active.

The second decoupling circuit can be an actively decoupled circuit. For example, the second decoupling circuit can include a switch (e.g. a controllable switch, a semiconductor switch, a PIN diode, etc.) that can be activated (e.g. ON and OFF) by a bias current (e.g. 5 volts DC). For example, the bias current is applied to the switch of the decoupling circuit to decouple or detune the second radio frequency coil.

For example, the coil assembly further includes a scaffold, wherein the first and second radio frequency coil elements are connected to the scaffold.

For example, the scaffold includes an internal surface and an external surface, and the first radio frequency coil element is arranged on the external surface of the scaffold and the second radio frequency coil element is arranged on the internal surface of the scaffold.

For example, the frequency interval is less than 35% of the second frequency.

For example, the frequency interval is less than 30% of the second frequency.

For example, the frequency interval is less than 25% of the second frequency.

For example, the frequency interval is less than 20% of the second frequency.

For example, the frequency interval is less than 15% of the second frequency.

For example, the frequency interval is less than 10% of the second frequency.

For example, a pair of the first and second spin species includes one of: 19F and 1H; 31P and 7Li; 27Al and 13C; 6Li and 170; 10B and 15N; 6Li and 9Be; 9Be and 17O; and 21Ne and 33S.

In some embodiments, the first and second spin species can be the same isotope, for example 19F, wherein for example the coil assemblies and resonance systems are used to identify 19F containing compounds having one or more 19F chemical shifts.

In some embodiments, the first and second spin species can be in different molecular environments, for example when a 19F containing compound is in a membrane bound versus free state.

For example, the coil assembly further includes at least one tuner for separately tuning each of the first and second radio frequency coil elements to a magnetic resonance detectable spin species. The coil assembly can also include a first tuner for tuning the first radio frequency coil element and a second tuner for tuning the second radio frequency coil.

For example, the coil assembly further includes a power means for powering the circuitry. The coil assembly can also include a controller for powering and/or controlling various parts of the circuitry.

In some embodiments, the coil assembly further includes a cover for covering the scaffold.

Also provided in another aspect is a holding assembly comprising one or more coil assemblies described herein and a holder for placing the subject or test sample. For example, a holding assembly includes a coil assembly as described herein and a holder for placing the subject or test sample.

For example, the holder can include a partially enclosed space for placing the subject, a portion thereof and/or the test sample.

The coil assemblies can be positioned for example at opposing sides for example to encircle the subject, a portion thereof and/or the test sample.

Also provided in another aspect is a magnetic resonance device optionally a magnetic resonance imaging (MRI) device comprising the holding assembly as described herein and a resonator connected to circuitry, the resonator including the second transmitter coil element configured for transmitting the second radio frequency signal for exciting the second spin species in the region of interest.

For example, the resonator or second transmitter is or includes a cylindrical detunable resonator.

For example, in some embodiments the device further includes a resonator tuner for tuning the second transmitter to one magnetic resonance detectable spin species.

Also provided in another aspect is a system, comprising the coil assembly, holding assembly or device described herein and further including a magnet and a magnet controller for controlling the homogeneity and stability of a magnetic field generated by the magnet.

For example, the magnet includes an opening for receiving the coil assembly and optionally the resonator. For example, the device further includes a receiver unit connected to the circuitry for receiving the second radio frequency signals from the second radio frequency coil element.

For example, the device and/or system further includes an imager that reconstructs electronic image representations from the received second radio frequency signals.

According to another aspect, there is disclosed a method of receiving magnetic resonance signals, including:

generating a magnetic field around a region of interest of a subject or test sample;

transmitting, with a first radio frequency coil element, a first radio frequency signal through the region of interest, said first radio frequency signal for exciting a first magnetic resonance detectable spin species;

transmitting, with a second transmitter coil element, a second radio frequency signal through the region of interest, said second radio frequency signal for exciting a second magnetic resonance detectable spin species in the region of interest, wherein
the first radio frequency signal and the second radio frequency signal are separated by a frequency interval,
the second magnetic resonance detectable spin species is modulated by the first magnetic resonance detectable spin species;

capturing, with a second radio frequency coil element, a magnetic resonance signal from the second magnetic resonance detectable spin species; and

optionally processing the captured magnetic resonance signal.

For example, the steps of transmitting with a first radio frequency coil element and capturing with a second radio frequency coil element, can be performed using a coil assembly and/or device described herein. In some embodiments, the step(s) of generating a magnetic field and/or transmitting with a second transmitter coil element can be performed with a device described herein.

In some embodiments, the first spin species is different from the second spin species.

The region of interest of a subject can be any region available for MRI, for example an organ such as brain, lungs, spines, intestines, muscle, or liver.

The test sample can for example be a tissue comprising cells for example growing on a tissue culture plate or in a 3D culture system. The test sample can also be a tissue comprising cells printed with a 3D printing system.

For example, the method further includes decoupling the second radio frequency coil element when the first radio frequency coil element transmits the first radio frequency signal.

For example, the method further includes decoupling the second transmitter coil element when the second radio frequency coil element receives the magnetic resonance signal.

For example, the method further includes decoupling the second radio frequency coil element when the first radio frequency coil element transmits and/or receives the magnetic resonance signal.

For example, processing the captured magnetic resonance signal includes filtering and amplifying the captured magnetic resonance signal.

For example, the method further includes converting the processed magnetic resonance signal into a digital signal to obtain a magnetic resonance digital signal.

For example, the method can include reconstructing and optionally displaying electronic image representations from the magnetic resonance digital signal.

For example, the second transmitter coil element can be included within a resonator configured for transmitting the second radio frequency signal for exciting the second spin species in the region of interest.

According to one aspect, there is disclosed a method for tracking of a compound in a subject or test sample, the method comprising:
a. introducing the subject or a test sample thereof into a holding assembly or a device, wherein the subject has been or will be administered the compound;
b. receiving magnetic resonance signals according to a method described herein, wherein the compound comprises at least one isotope of the first spin species, optionally 13C, 15N, 19F or 31P;
c. optionally processing the captured magnetic resonance signal to obtain an image; and
d. determining the position or positions of the compound or a metabolite thereof in the subject or test sample from the processed captured magnetic resonance signal.

In some embodiments, the method further comprises quantifying the amount of compound and/or metabolite determined at the one or more positions.

In some embodiments, the at least one isotope is 19F.

The region of interest of a subject can be any region available for MRI, for example an organ such as brain, lungs, spines, intestines, muscle, or liver.

For example, when the region of interest is the brain, the subject can be introduced into the holding assembly such that the coil assembly is or coil assemblies are situated around the head of the subject.

For example, localization/spatial information of the spin species is accomplished using a spin-echo or gradient-echo sequence preceded with a magnetization transfer (MT) pulse allowing magnetization from 19F to 1H.

For example, the method further includes producing an image, optionally wherein the level of compound is indicated by colour intensity or colour difference in the image.

In some embodiments, the subject is a mammal. For example, the mammal can be a rat, a mouse, dog, guinea pig, rabbit, cat, pig, a livestock animal or horse. For example, the mammal can be a human.

In some embodiments, the test sample is a tissue and/or comprises cells, for example a 2D or 3D cell culture, optionally a 3D printed tissue like structure or organ.

Such methods can be used to detect the in vivo localization and can be used for quantification of a compound for example as shown in the Examples.

For example, the compound is a drug for treating a disease. For example, the compound is a diagnostic agent.

For example, the method can be used for monitoring localization of the compound over a selected time interval.

The method can be used to monitor and/or optimize treatment regimens and doses. For example one or more doses and/or a drug of a treatment regimen can be administered to the subject. After a suitable time, the subject (or a region of interest) can be imaged and optionally reimaged using a coil assembly, holding assembly, device or MR system described herein. If a desired amount of the drug is detected, the treatment regimen can continue and optionally continue to be monitored. If a desired amount is not detected, the treatment regimen can be altered by increasing or decreasing the amount or frequency of administration of the drug.

No embodiment described below limits any claim and any claim may cover methods or apparatuses that differ from those described below. The claims are not limited to apparatuses or methods having all of the features of any one coil assemblies devices or methods described below or to features common to multiple or all of the apparatuses described below. It is possible that an apparatus or method described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

### BRIEF DESCRIPTION OF THE DRAWINGS

The accompanying figures, which are incorporated in and constitute an integral part of the specification, illustrate various example coil assemblies, holding assemblies, devices, systems, methods, and example embodiments of various aspects of the invention and are meant to provide additional explanation so that the disclosure can be better appreciated by those of ordinary skill in the art. Additionally, elements in the figures are not drawn to scale.

FIGS. 1A and 1B show general schematics for MR systems, according to exemplary embodiments.

FIGS. 2A and 2B show general schematics for other MR systems, according to exemplary embodiments.

FIGS. 3A and 3B show general schematics for alternative MR systems, according to exemplary embodiments.

FIG. 4A shows the top-down view of a representative RF coil assembly, according to one example.

FIGS. 4B and 4C show representative RF coil assemblies, according to other examples.

FIGS. 4D and 4E show coil assemblies, according to other examples.

FIG. 5 shows the bottom-up view of the RF coil assembly of FIG. 4A.

FIG. 6 shows the side view of the RF coil assembly of FIG. 4A.

FIG. 7 shows the end-on view of the RF coil assembly of FIG. 4A.

FIGS. 8A and 8B shows the circuitry diagram for the representative RF coil assembly of FIG. 4A.

FIG. 9 shows the top view of part of a holding assembly, according to one example.

FIG. 10 shows the side view of part of the holding assembly of FIG. 9.

FIG. 11 shows the top view of the holding assembly of FIG. 9.

FIG. 12 shows an exploded view of the RF coil assembly of FIG. 4A with the holding assembly of FIG. 9 together with a detunable resonator, according to one example.

FIG. 13 shows the RF coil assembly positioned on top of the holding assembly, according to one example.

FIG. 14 shows the cover positioned on top of the holding assembly after installation, according to one example.

FIG. 15 shows the components of FIG. 12 positioned within the cavity of the detunable resonator, according to one example.

FIG. 16 shows the components of FIG. 15 positioned into the cavity of a magnet of an MR system, according to one example.

FIG. 17 shows results from an MRI experiment using the coil assembly disclosed herein, according to one example.

FIG. 18 shows a pulse sequence, according to one example.

### DETAILED DESCRIPTION OF THE DISCLOSURE

The foregoing and other aspects of the present disclosure will now be described in more detail with respect to other embodiments described herein. It should be appreciated that the disclosure can be embodied in different forms and should not be construed as limited to the embodiments set forth herein. Rather, these embodiments are provided so that this disclosure will be thorough and complete, and will fully convey the scope of the disclosure to those skilled in the art.

The terminology used in the description of the disclosure herein is for the purpose of describing particular embodiments only and is not intended to be limiting. As used in the description of the disclosure and the appended claims, the singular forms “a”, “an” and “the” are intended to include the plural forms as well, unless the context clearly indicates otherwise. Additionally, as used herein, the term “and/or” includes any and all combinations of one or more of the associated listed items and may be abbreviated as “/”.

Unless otherwise defined, all technical and scientific terms used herein have the same meaning as commonly understood by one of ordinary skill in the art to which this disclosure belongs. All publications, U.S. patent applications, U.S. patents, international patent publications, journal articles, monographs, books, and other references cited herein are incorporated by reference in their entireties.

The term “balun” refers to an electrical device that converts between a balanced signal (two signals working against each other where ground is irrelevant) and an unbalanced signal (a single signal working against ground or pseudo-ground) or vice versa. A balun can have many forms and includes devices that also transform impedances. Such transformer baluns can also be used to connect transmission-lines of differing impedance.

The term “coil assembly” refers to a structure having coil element portions, electrical conductor portions, capacitive and/or inductive components, circuitry portions, and any other suitable electrical components, scaffolding and/or protective components such as a cover for covering the scaffold. The term “coil element” refers to a resonant wire component of the coil assembly. The coil element or a portion thereof can have a particular shape such as a loop, spiral, saddle or helix.

The term “couple” refers to the interaction between two nuclei or two RF coil elements. When the two nuclei are the same, this is referred to as “homonuclear”, while if they are different, this is referred to as “heteronuclear.” Likewise, the terms “coupling” or “coupled” refer to actions that describe this process.

The term “decouple” or “detune” refers to reduce/null interference between the coupling of at least one nuclei or RF coil elements and at least one nuclei or RF coil elements. Likewise, the terms “decoupling” or “decoupled” refer to actions that result in this process.

One possible method to protect a RF coil element and its associated electronics is to decouple the receive coil elements when RF is being transmitted by an MR apparatus to create the B1 magnetic field. This decoupling may be active or passive.

The term “gradient echo” when referring to a pulse sequence indicates a single RF excitation pulse, followed by a gradient reversal to generate transverse magnetization.

The term “hyperpolarization” refers to the forced alignment of all (or most) nuclei in the primary magnetic field (B0) in the same direction. This increase in polarization enhances the MR signal from a particular region of interest of a subject or test sample. It is particularly useful for those nuclides of low natural abundance or low sensitivity. One technique used for hyperpolarization is dynamic nuclear polarization (DNP), which is of particular interest for metabolism studies because it has the potential to dramatically increase the sensitivity to molecules containing 13C nuclei. The term “lumped-element circuits” refers to a circuit with physical dimensions such that voltage across and current through conductors connecting the elements is invariant. The lumped element model of electronic circuits makes the simplifying assumption that the attributes of the circuit, resistance, capacitance, inductance, and gain, are concentrated into idealized electrical components; resistors, capacitors, and inductors, etc. joined by a network of perfectly conducting wires.

The term “modulation” or “modulating” refers to an increase, transfer, facilitation, upregulation, activation, inhibition, decrease, blockade, prevent, delay, desensitization, deactivation, down regulation, or the like, of a process or mechanism. For example, modulation of a second magnetic resonance detectable spin species by a first magnetic resonance detectable spin species refers to a perturbation from the first MR detectable spin species to the second MR detectable spin species.

The term “PIN diode” refers to a diode with a wide, undoped intrinsic semiconductor region between a p-type semiconductor and an n-type semiconductor region in which both regions are typically heavily doped because they are used for ohmic contacts.

The term “pulse” refers to the creation of the perturbing magnetic field (B1) used to generate magnetic resonance signals of a specific frequency, the length of which can be varied. A pulse can be performed using a detunable resonator or a radio frequency coil element used as a transmitter.

The term “pulse sequence” refers to a series of pulses employed simultaneously or sequentially to obtain a particular magnetic resonance outcome. Pulse sequences are used to perturb one or more spin species in a specific manner. When a pulse sequence is utilized for the same spin species, it is termed homonuclear, while when the spin species are different, it is termed heteronuclear. Such pulse sequences range from general purpose single-pulse experiments to complex, highly sophisticated experiments that target specifically interacting nuclei. Representative examples of pulse sequences, which are often referred to using the acronyms indicated, are the following: chemical exchange saturation transfer (CEST), correlation spectroscopy (COSY), difference nuclear Overhauser enhancement (DNOE), distortionless enhancement by polarization transfer (DEPT), dynamic nuclear polarization (DNP), exchange spectroscopy (EXSY), exclusive correlation spectroscopy (ECOSY), heteronuclear decoupling, heteronuclear multiple-bond correlation spectroscopy (HMBC), heteronuclear single-quantum correlation spectroscopy (HSQC), incredible natural-abundance double-quantum transfer experiment (INADEQUATE), insensitive nuclei enhanced by polarization transfer (INEPT), magnetization transfer (MT), nuclear Overhauser enhancement (NOE), nuclear Overhauser effect spectroscopy (NOESY), rotating frame nuclear Overhauser effect spectroscopy (ROESY), total correlation spectroscopy (TOCSY). Still other pulse sequences will be known to those in the art.

The term “saddle coil element” refers to a coil element that is arranged along the perimeter of a surface curved over a cylinder wall. Said surface can be rectangular in shape when flattened, for example, but also be any kind of polygon or can have rounded rather than sharp corners.

The term “solenoid coil element” is understood to be a coil element, the windings of which run substantially in the shape of a helical line with a slight incline along a lateral surface of a cylinder.

The term “spin-species” as used herein includes the same or different nuclides, where their spins can be considered separate and distinct.

The term “spin-echo” when referring to a pulse sequence in its simplest form indicates a 90° RF excitation pulse with refocusing during the echo time by using a 180° RF pulse.

The term “gradient-echo” when referring to a pulse sequence in its simplest form indicates a 90° RF excitation pulse with a rapid reversal gradient followed by a smaller amplitude opposite polarity gradient during the readout to generate the “gradient-echo”.

The term “gyromagnetic ratio” (symbolized as γ) refers to an inherent characteristic of each nuclide, a constant that defines the relationship between resonant frequency and field strength (Table 1). Negative values for γ mean that direction of nuclear spin is opposite to that of 1H.

TABLE 1
Gyromagnetic Ratios for Representative Nuclides

| | Gyromagnetic ratio (γ) |
|---|---|
| Nuclide | (MHz/T) |
| ¹H | 42.58 |
| ²H | 6.54 |
| ³He | −32.43 |
| ⁷Li | 16.55 |
| ¹³C | 10.71 |
| ¹⁴N | 3.08 |
| ¹⁵N | −4.32 |
| ¹⁷O | −5.77 |
| ¹⁹F | 40.05 |
| ²³Na | 11.26 |
| ²⁷Al | 11.10 |
| ²⁹Si | −8.47 |
| ³¹P | 17.24 |
| ⁵⁷Fe | 1.38 |
| ⁶³Cu | 11.32 |
| ⁶⁷Zn | 2.67 |
| ¹²⁹Xe | −11.78 |

The term “resonant frequency” (or “Larmor frequency”) refers to a radiofrequency value determined by a combination of nuclear characteristics and the strength of the magnetic field. The Larmor frequency (v₀) in units MHz is given by the equation: v₀=γB0, where γ represents the gyromagnetic ratio and B0 is the primary magnetic field in Tesla (T).

The term “subject” as used herein denotes any animal, preferably a mammal including a human. Examples of subjects include humans, non-human primates, rodents, including mice and rats, guinea pigs, rabbits, sheep, pigs, goats, cows, horses, dogs and cats. Avian and reptile animals are also included.

1. Magnetic Resonance Coil Assemblies, Holding Assemblies, Devices and Systems

As described herein, the magnetic resonance (MR) coil assemblies, holding assemblies, devices and systems of the disclosure are comprised of multiple components, including for example at least two radio frequency coil elements, decoupling means, and optionally a holding assembly or for example a main magnet, a detunable resonator, at least two radio frequency coil elements, decoupling means, a holding assembly, and a computing device/controller. In addition, a variety of electronic elements and drive circuitry, plus various means for specific functions, are employed with one of more of these components, so that they can be operated in a manner appropriate for the conduct of MR studies of test samples. These components can be separate from or integrated with each other in full or in part. Further, the components can be arranged and configured in numerous ways as will be appreciated by those in the art.

In a preferred embodiment, the MR system is a magnetic resonance spectroscopy (MRS) instrument. In another preferred embodiment, the MR system is a magnetic resonance imaging (MRI) instrument.

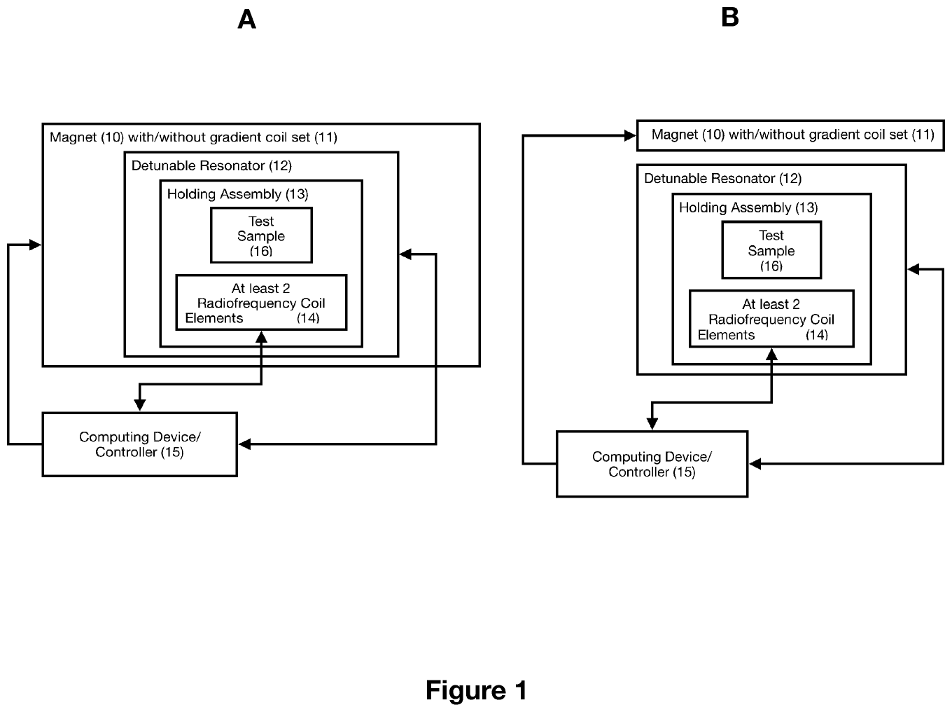

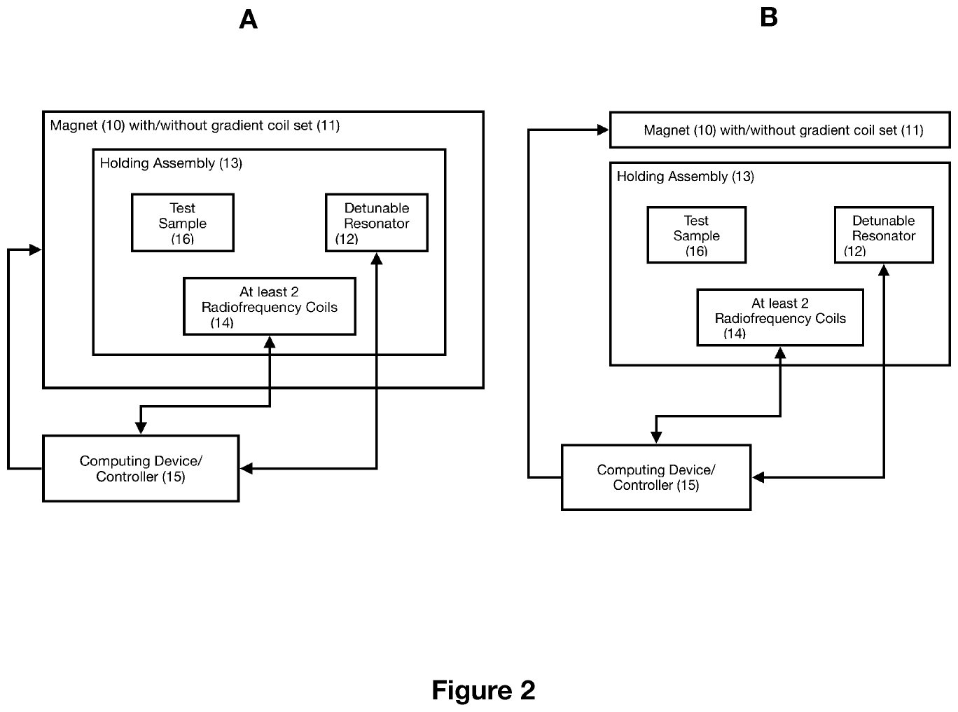

In the representative examples shown in FIGS. 1A, 1B, 2A and 2B, a block diagram of typical components of a magnetic resonance (MR) system of the disclosure is presented, which comprises a main magnet (10) with or without a gradient coil set (11), detunable resonator (12), holding assembly (13), at least two (2) radio frequency (RF) coil elements (14) that act as transmitter, receiver, or both, computing device or controller (15), along with the subject or test sample (optionally referred to as test sample for simplicity) (16). In addition to these principal components, there will also be other magnetics components, such as shim coil elements, a power source, one or multiple power supplies, one or multiple pre-amplifiers, or power management devices, and connecting circuitry between the various components. As shown in FIGS. 1A, 1B, 2A and 2B, this specifically includes connections of 15 to 10/11, 12 and 14. Although a MR system of the disclosure will generally include these components, the implementation of these components for a particular MR system may differ somewhat, as discussed in further detail below.

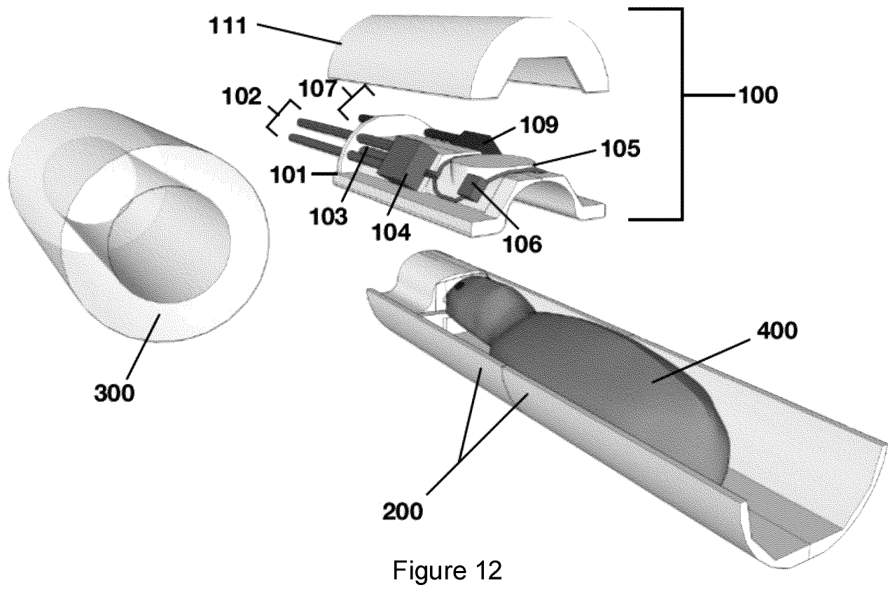

For example, the radio frequency detunable resonator (12) may be situated or insertable completely within the cavity of the magnet (10), with or without the set of gradient coil set (11), along with the holding assembly (13), as illustrated in FIG. 1A. In turn, 13 completely contains the RF coil elements (14) and the test sample (16). Alternatively, 12, together with 13, may only be partially within 10/11, or completely outside of 10/11 but within its resulting magnetic field, as illustrated in FIG. 1B. In this alternative configuration as well, 14 and 16 reside completely within the holding assembly (13). Similarly, the connections of 15 to 10/11, 12 and 14 remain. Although not shown in FIGS. 1A and 1B, 13 may also only be partially inserted into the cavity of the resonator (12). Proper positioning of the RF coil elements relative to the test sample can be important in all these configurations in order to obtain the most useful information. The coil elements should effectively be close in proximity, in three dimensions, to the region of interest of the subject or test sample or, the specific region of the subject or test sample that is to be investigated.

An alternative configuration is provided in the representative MR system shown in FIGS. 2A and 2B. In this example, the block diagram indicates that the detunable resonator (12) is within the holding assembly (13) along with the RF coil elements (14) and test sample (16). This assembly is then either entirely within the main magnet (10) with or without the set of gradient coil set (11) as in FIG. 2A or partially within 10/11, or completely outside of 10/11 but within its resulting magnetic field, as in FIG. 2B. Although not shown in FIGS. 2A and 2B, the specific arrangement of 12, 14 and 16 within 13 is not specified, but 12 and 14 can be positioned in relation to 16 such that the appropriate experiment can be effectively performed as will be evident to one skilled in the art.

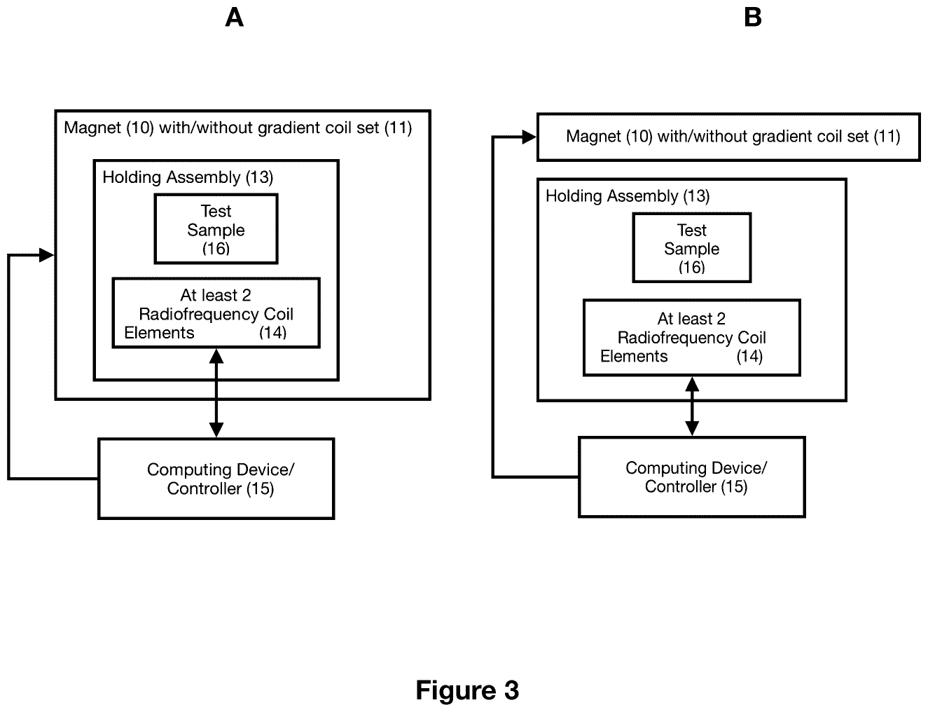

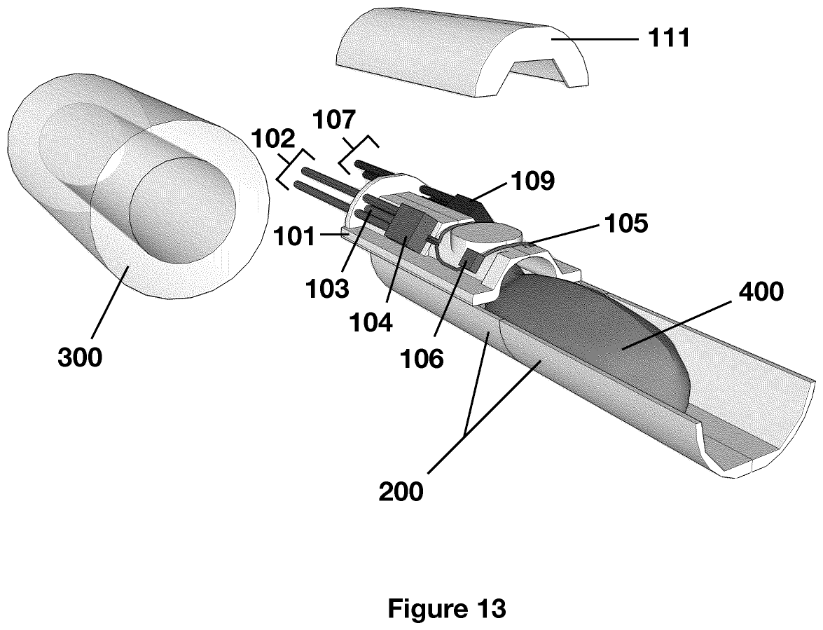

Yet another configuration for a MR device or system is presented in FIGS. 3A and 3B, in this case, the detunable resonator (12) is not included, but otherwise is analogous to that in FIG. 1. The holding assembly (13), may be situated completely within the cavity of the magnet (10), with or without the set of gradient coil set (11) as is illustrated in FIG. 3A. In turn, 13 completely contains the RF coil elements (14) and the test sample (16). Alternatively, 13, may only be partially within 10/11, or completely outside of 10/11 but within its resulting magnetic field, as illustrated in FIG. 3B. In this alternative configuration as well, 14 and 16 reside completely within 13. Connections of the computing device or controller (15) to 10/11 and 14 are present in both FIGS. 3A and 3B. Although not shown in FIGS. 3A and 3B, 14 and/or 16 may also only be partially inserted into the cavity of the holding assembly (12). Regardless, the same considerations regarding proper positioning of the RF coil elements relative to the sample as previously described remain relevant in both these configurations.

In some embodiments, the device or system comprises multiple coil assemblies each comprising at least two radio frequency coil elements and decoupling means, suitably positioned relative to the test sample or subject, to obtain useful information from for example different areas of an organ such as the brain, lungs, spines, intestines, muscle or liver.

It will be appreciated by those in the art that the MR coil assemblies, holding assemblies, devices and systems presented in the Figures are meant to be representative only and may have one or more other components of any suitable type in addition to or instead of the components shown as provided for in the following detailed description. Additional configurations and methods for using the MR assemblies, devices and systems of the disclosure are presented in the Examples.

A. Magnet

This magnet will typically have, but not limited to, at least a partially enclosed cavity, or bore, within which other components and/or test samples can be placed. As well, it is connected to one or a combination of components that permit control of the strength and homogeneity of the magnetic field. The primary purpose of the main magnet is to create a stable and static primary magnetic field, B0, which functions to magnetize the test sample. In addition to the field strength, the primary magnetic field can be very homogeneous within the region where the test sample is placed. Fluctuations, or inhomogeneities, in the field strength cause MR signals to degrade leading to poor quality spectra (broad lines) and imaging scans (spatial distortions, poor resolution).

Therefore, the main magnet usually has at least one, often multiple, shim coil elements for correcting the inhomogeneities in the primary magnetic field, such as can be caused by the materials comprising the MR system, changes in the local environment, and even insertion of the test sample itself. Two types of shim coil elements, passive and active, can be employed. For the former, small metallic or ferromagnetic pieces, such as pellets, are affixed to various specific locations around the main magnet, including within the cavity, to improve homogeneity. With active shim coil elements, current flowing through them generates a magnetic field that can be utilized to correct B0 inhomogeneities. Such coil elements can be individually adjusted based on the amount of current permitted to flow through them in order to restore field homogeneity. Such coil elements can be integrated within the same housing as the main magnet or otherwise positioned to be able to influence B0. A field homogeneity of 1 part per million (ppm) or 1 part per billion (ppb) can be achieved employing the shim coil elements. The required level of homogeneity is dependent on the specific demands of the application, as can be determined by one skilled in the art. In some embodiments, the shimming process using such coil elements is automated. Although passive shim coil elements are self-contained, active shim coil elements typically require their own power supplies and control circuitry. In some embodiments of the disclosure, only active shim coil elements are present, while in other embodiments, only passive shim coil elements are present. In still other embodiments, both active and passive shim coil elements are present.

The strength of a magnet is most often given in Tesla (T) with 1 T=10,000 gauss. Magnets employed in MRS systems are often referred to using a frequency rather than the field strength. For example, a system containing a 21.1T magnet may be referred to as a 900 MHz system, corresponding to the resonant frequency of 1H in that field. Non-limiting representative magnet strengths utilized in the MR systems of the disclosure are 1.4T (60 MHz), 2.35T (100 MHz), 3T (127 MHz), 4.1T (175 MHz), 4.7T (200 MHz), 7.05T (300 MHz), 9.4T (400 MHz), 11.75T (500 MHz), 14.1 (600 MHz), 16.5T(700 MHz), 17.6T (750 MHz), 18.8T (800 MHz), 20.0T (850 MHz), 21.1T (900 MHz), 22.3T (950 MHz), 23.5T (1,000 MHz), 24.0T (1,020 MHz, J. Magn. Res. 2015; 256, 30-33). For example, current main magnet strengths for MRI systems, such as in routine clinical use for human subjects, can range from 0.06T-4.0T, while those employed for research purposes for human subjects can extend this to 7.0-10.5T, while with non-human subjects can be as high as 21.1T. As another example, MRS systems for structural determination of organic and inorganic substances possess field strengths from 2.35T-23.5T with higher fields typically required in order to ascertain the structures and configurations of larger, more complex molecules, such as proteins.

In preferred embodiments, superconducting magnets are utilized for the main magnet as they can attain very high field strengths with excellent stability. In such magnets, the electrical current required to power it flows without resistance; hence, once charged, no outside energy source is needed to maintain the field strength. However, achieving and maintaining that superconducting state requires extremely low temperatures, near absolute zero, so an appropriate substance, typically liquid helium, is employed to maintain the magnet at such temperatures, for example, approximately 1.7-4.0K (−271.5 to −269.2° C., −456.6 to −452.5° F.). To assist in maintaining this superconducting state and provide thermal insulation, the main magnet is placed inside at least one cryostat. A secondary cryostat, typically containing liquid nitrogen, can be employed to further insulate the superconducting main magnet. In certain cases, a cryocooler unit can be employed to recondense helium vapor back into the liquid state. Despite these protections, the helium does slowly dissipate, so additional liquid helium can be regularly provided. To avoid this, as well as due to the increasing cost and limited availability of liquid helium, the main magnet can also be cooled directly using a cryogenic cooling unit or system. These cryocoolers operate much like a conventional air-conditioning unit, relying on the compression and expansion of a fixed volume of gas under pressure in a closed, self-contained circuit, although in this case typically employing helium gas.

Main magnets utilized in these superconducting systems can be made using alloys containing rare-earth elements such as niobium, in particular niobium-titanium (NbTi) and niobium-tin (Nb3Sn) alloys, generally provided in a solenoid coil geometry. Active shim coil elements used with a superconducting magnet can themselves be superconducting if they are located within the cryostat, or, alternatively, can be resistive if attached to a room-temperature component of the system, such as within the cavity where the test sample is placed.

In other embodiments, permanent main magnets can be used to provide the primary magnetic field. However, their size, weight, weaker field strengths, limited precision and stability, does restrict their overall utility. Nonetheless, they have been employed in benchtop MRS systems used for chemical analysis, reaction monitoring and quality control experiments, and smaller MRI scanning systems due to their lower overall costs. These permanent magnets are made from ferromagnetic materials, such as alloys containing the rare-earth element neodymium, for example NdFeB, an alloy of neodymium, iron and boron. The field strength of such magnets is typically lower (0.3-1.5T) and sensitive to fluctuation with temperature, although use of appropriate shielding of the magnet can be employed to rectify this situation.

In another embodiment, the main magnet can be an electromagnet. The magnetic field is produced by an electric current in this type of magnet, but disappears when the current is halted. The most common electromagnet comprises conducting wire wound into a coil element.

The main magnet may be any suitable type or combination of magnetics components that can generate the desired main magnetic field, B0. As well, the main magnet can be a variety of shapes, including, but not limited to, cylindrical, planar, C-shaped and box-shaped. In particular embodiments, a superconducting main magnet has a cylindrical shape, while in other embodiments, a permanent magnet is C-shaped.

The main magnet may or may not possess a gradient coil set, typically composed of one or more gradient coil elements composed of multiple wire loops or thin conductive sheets. Each such coil element is an electromagnet integrated with the MR main magnet and most often situated between it and the test sample. When present, three gradient coil elements are typically employed, with each coil element creating a gradient magnetic field that varies linearly along one of three substantially orthogonal dimensions; hence, these are termed the x-, y-, and z-gradients. As such, this secondary magnetic field created results in localized distortions in the primary magnetic field and permits spatial encoding of the MR signal. Such gradient magnetic fields can be pulsed, as well as varied over a time course. These gradient coil elements create the magnetic field in a predictable manner in space, so they can be particularly useful for three-dimensional and imaging applications. In other words, the gradient magnetic fields permit localization and detection of MR signals both across an entire subject or test sample or in only a specific region of the subject or test sample. Since the resonance frequency of a particular substance is directly proportional to the strength of the applied magnetic field, nuclei in the sample will have resonance frequencies dependent on their location in the field.

Gradient coil elements of various compositions and a diversity of construction designs can be included in the MR systems of the disclosure. For example, a gradient coil element can be composed of wires wrapped around a fiberglass cylindrical form and coated with epoxy resins. With superconducting magnets, multiple thin metallic strips or large copper sheets etched into complex patterns and applied to a cylinder structure can be utilized for the gradient coil elements.

In a preferred embodiment, the MR system is a MRI instrument with such gradients for localization. In another preferred embodiment, the MR system is a MRS instrument with gradients for localization. In still another preferred embodiment, where such gradient coil elements are not present the MR system is a MRS instrument without gradients for localization.

B. Resonator

Another component of MR devices and systems includes for example the radio frequency detunable resonator, which has the ability to be tuned to the frequency of one particular magnetic resonance detectable spin species. The resonator generates the B1 magnetic field that is used to perturb the test sample. Such perturbation preferably occurs in a homogeneous manner across the entire subject or test sample, in contrast to the use of the gradient coil elements that vary the B₀ magnetic field in defined spatial regions as already described. B1 is typically applied perpendicular to the primary magnetic field (B₀) as this arrangement maximizes the resulting signal, although different angles can also be used depending on the application. B₁ is typically only active for a defined, usually short time period (for example, 1-5 milliseconds), or pulse. Depending on the nature of the analysis being performed, this perturbation may occur over the entire subject or test sample or only over a specific region of interest, for example a particular organ or subject body part or portion of a material. The RF detunable resonators can be composed of metal alloys, in particular with rare earth metals, ferroelectric functional materials, such as BaTiO₃, or mixtures of these.

In certain embodiments, at least one of the radio frequency coil elements is tuned to the same frequency as the radio frequency detunable resonator and a separate resonator component is not present (for example, see FIG. 3). However, when present, a means of tuning the resonator to a specific frequency also is necessary, as is drive circuitry connected to that resonator. In some embodiments, the detunable resonator is a volume coil element. In other embodiments, the means for tuning the resonator is combined with, integrated with, or the same as other means of controlling, tuning, recording or directing that are involved in the MR device or system. In still other embodiments, the drive circuitry for the resonator is combined with, integrated with, or the same as other drive circuitry that are involved in the MR device or system, including the drive circuitry associated with the radio frequency coil elements. In further embodiments, the detunable resonator fits at least partially into the magnet, while in alternative embodiments, the resonator fits entirely into the magnet.

Representative examples of resonators encompassed by the disclosure include those described in: U.S. Pat. Nos. 4,641,097; 5,194,811; 5,202,635; 5,212,450; 5,886,596; 6,100,691; 6,366,093; 6,969,992; 7,119,541; 9,035,655; 9,939,502; U.S. Pat. Publ. No. 2006/0012370; PCT Intl. Pat. Publ. Nos. WO 92/08145; WO 92/13283; Magn. Reson. Med. 1994, 32(2), 206-218; J. Magn. Reson. B. 1995, 107(1), 19-24; Magn. Reson. Med. 1997, 38(1), 168-172; J. Magn. Reson. 2008, 191(1), 78-92; NMR Biomed. 2001, 14(3), 184-191; Magn. Res. Imaging 2001, 19, 1339-1347; Magn. Reson. Med. 2002, 47(2), 415-419; Magn. Reson. Med. 2002, 47(3), 579-593; Magn. Reson. Med. 2002, 47(5), 990-1000; J. Neurosci. Methods 2004, 132(2), 125-135; Conf. Proc. IEEE Eng. Med. Biol. Soc. 2007, 3884-3885; NMR Biomed. 2009, 22(9), 908-918; J. Magn. Reson. 2012, 217, 10-18; Magn. Reson. Med. 2012, 68(4), 1325-1331; Phys. Med. Biol. 2012, 57(14), 4555-4567; IEEE Trans. Med. Imaging 2013, 32(6):1081-1084; NMR Biomed. 2013, 26(11), 1555-1561; IEEE Trans. Biomed. Eng. 2014, 61(2), 327-333; PLoS One 2015, 10(3), e0118892J; J. Korean Phys. Soc. 2016, 68(7), 908-913; IEEE Trans. Biomed. Eng. 2016, 63(11), 2390-2395; Sci, Rep. 2017, 7(1), 2038; PLoS One 2018, 13(2), e0192035; Magn. Reson. 2018, 291, 47-52; Magn. Reson. Med. 2018, 80(1), 361-370; Magn. Reson. Med. 2018, 80(3), 1005-1019.

C. Radio Frequency Coil Elements and Coil Assemblies

As mentioned previously, the radio frequency (RF) coil elements are components of the coil assemblies, holding assemblies, devices and MR systems, which in general can function to perturb spin species in the test sample and/or then detect the resulting MR signal therefrom. For the MR assemblies, devices and systems of the present disclosure, at least two RF coil elements are employed. In some embodiments, the MR assemblies, devices and systems have two RF coil elements, while in other embodiments, the assemblies, devices and systems have more than two such elements. When used as a transmitter, the RF coil element is employed to produce an oscillating radio frequency magnetic field (B1) at the resonance frequency of a magnetic resonance detectable active spin species, thereby perturbing the spin states of that nuclide, resulting in the detection of an RF signal as the spin species return to its original equilibrium state. Other considerations for the generation of B1 using the RF coil elements are the same as already detailed for the detunable resonator in the previous section. The transmitter RF coil element can be configured to generate any suitable type of RF pulse. When utilized as a receiver, the RF coil element receives the radio frequency signal from spin species during relaxation back to its original state. As will be appreciated by those in the art, an RF coil element can be a transmitter, a receiver, or both (a transceiver), dependent on having the proper configuration of circuitry and means of control to be used for the indicated function. In certain embodiments, a switch is used to select whether a RF coil element operates as a transmitter or receiver. Further, if at least two coil elements are transceivers, the pulse sequences require changes to be able to handle the non-homogeneous RF field, as is within the capabilities of those in the art.

Coil assemblies that transmit may be designed to handle more power (e.g. larger capacitors/inductors) relative to a coil assembly that is a receive only coil, which would be designed have high Q (less lossy).

Therefore, in some embodiments, at least one of the RF coil elements is a transmitter and at least one of the RF coil elements is a receiver. In further embodiments, the number of RF coil elements that are transmitters equal the number of RF coil elements that are receivers. In other embodiments, the number of RF coil elements that are transmitters is greater than the number of RF coil elements that are receivers, while in still other embodiments, the number of RF coil elements that are transmitters is less than the number of RF coil elements that are receivers. In further embodiments, at least one of the RF coil elements is a transceiver. In such a case, the RF coil element assumes the function of the detunable resonator, so that a separate component for that purpose is not necessary as is illustrated in FIG. 3.

Similar to the detunable resonators, the RF coil elements can be composed of metal alloys, in particular with rare earth metals, ferroelectric functional materials, such as BaTiO3, or mixtures of these. Numerous shapes, configurations, designs, and materials of radio frequency coil elements for a variety of applications have been described, including, but not limited to, circular coil elements, surface coil elements, saddle coil elements, birdcage coil elements, nested coil elements, transverse electromagnetic (TEM) coil elements, slotted tube coil elements, slotted elliptical tube coil elements, and those described in U.S. Pat. Nos. 4,797,617; 4,799,016; 5,184,076; 5,990,681; 7,081,753; 7,508,212; U.S. Pat. Publ. No. 2018/0003782; Magn. Reson. Imaging 1994, 12, 1079-1087; Concepts Magn. Res. 1997, 9, 195-210; J. Magn. Reson. Med. 1997, 38, 726-732; J. Magn. Reson. 1998, 131(1), 32-38; Magn. Reson. Imaging 1999, 77, 783-789; Magn. Reson. Med. 2002, 47, 579-593; J. Neurosci. Methods 2004, 132, 125-135; Brazilian J. Physics 2006, 36(1A), 4-8; J. Phys. Med. Biol. 2007, 52, 4943-4952; Microwaves and RF 2007, 46(11), 92-98.

In an embodiment, at least one of the RF frequency coil elements is a surface coil element. Surface coil elements provide a very high sensitivity over a relatively small region of interest, such as a certain portion of a heterogeneous chemical sample or a particular subject body part. Often, such coil elements are single or multi-turn loops, so that they can be easily placed in a particular location or molded/sized to fit the test sample area. In a particular embodiment, at least one of the RF frequency coil elements is a saddle coil element.

As noted previously, proper positioning of the RF coil elements relative to the sample can be important in order to obtain the most useful information. The coil elements should effectively be in close proximity, in three dimensions, to the subject or test sample or, the specific region of the subject or test sample to be investigated. If necessary, the size and shape of the coil elements can be adjusted in order to provide optimal interactions with any given region of interest.

In some embodiments, the at least two RF coil elements are the same size. The meaning of “same” in this context is within 1% of the size of the others. In some other embodiments, the at least two coil elements are within 1-5% of the same size. In other embodiments, the at least two coil elements are within 5-10% of the same size. In still other embodiments, the at least two coil elements are within 10-25% of the same size. In still further embodiments, the at least two coil elements are not within 25% of the same size.

The RF coil element is attached to circuitry, optionally drive circuitry, comprised of wires or other conducting material, capacitors, including parallel capacitors, inductors, resistors of various types appropriate for the application as will be known by those in the art. Depending on the configuration as a transmitter or receiver, this could include power supplies, pre-amplifiers, and other elements necessary for the desired function. In selected embodiments, each RF coil element can have active or passive modes of decoupling to minimize interactions with other nearby RF coil elements.

A coil element with active detuning (or decoupling) can comprise a drive circuit. A coil element with passive detuning (or decoupling) can include circuitry (such as a parallel capacitor and/or inductor, etc.) to detune the coil permanently from the frequency used on the passive decoupling component.

In certain embodiments, the radio frequency coil elements are attached in some manner to a scaffold and/or the holding assembly, such as with glue or epoxy, or by securing with non-magnetic or non-metallic fasteners or into specific indentations prepared for the coil elements in the scaffold and/or holding assembly. As well, the RF coil elements can be unattached to the scaffold and/or holding assembly and instead affixed to another part of the system.

D. Decoupling Element

Associated with the RF coil elements of the disclosure is a decoupling element. In some embodiments, the decoupling element is used for passive decoupling of one of the RF coil elements from at least one of the other RF coil elements. In other embodiments, the decoupling component is used for active decoupling of one of the RF coil elements from at least one of the other RF coil elements. Likewise, in preferred embodiments, the decoupling element is integrated with the RF coil element. In other embodiments, the decoupling element is separate from the RF coil element.

The present disclosure provides an improved arrangement to decouple RF coil elements in a MR assembly, device or system. An advantage resides in the ability to decouple RF coil elements of any type regardless of the resonant frequency at which they are operating. Indeed, the particular decoupling components used within the MR assemblies, devices and systems of the disclosure permit a very close spatial arrangement of RF coil elements of the same, or nearly the same frequency, such as 1H and 19F, to be achieved. Such an arrangement of the RF coil elements in conjunction with the integrated decoupling mechanisms enables MR assemblies, devices and systems to obtain magnetic resonance data from test samples, including living subjects, that are otherwise impractical.

A MR signal from a given nucleus can be affected by adjacent or nearby spin species of the same (homogeneous coupling) or different (hetereogeneous) nuclides. Although such “coupling” of signals often can provide useful information on the structure of an organic or inorganic substance, it can also adversely affect the signal strength and signal-to-noise ratio, as well as complicate the analysis of the signal. Such spin-spin interactions also can have detrimental effects on the integrity of components of the system, in particular the RF coil elements, the electronic circuitry, and the test sample itself. For this reason, it can be advantageous to prevent this coupling, i.e. decoupling, through incorporation of hardware elements into the system configuration, specific circuitry arrangements, and the use of particular pulse sequences and/or processing algorithms. A variety of different approaches have been reported for MR decoupling, including U.S. Pat. Nos. 6,414,488; 6,504,369; 6,747,452; 7,932,721; 8,049,504; 8,138,762; 8,380,266; 8,390,287; 9,069,048 9,869,732; U.S. Pat. Publ. Nos. 2007/0085540; 2018/0074140; PCT Intl. Pat. Publ. Nos. WO 2007/124247; WO 2008/032098; WO 2009/081359; WO 2010/073145; WO 2014/096997; J. Magn. Reson. 1987, 125, 178-184; J. Magn. Reson. 1979, 34, 425-433; J. Magn. Reson. 1997, 125, 178-184; Magn. Reson. Med. 2014, 72, 584-590; Magn. Reson. Med. 2015, 73, 894-900; Magn. Reson. Med. 2016, 75, 954-961; Sci. Reports 2018, 8, 6211

Some of these described elements are particularly suitable for decoupling specific nuclei from each other, such as 1H-13C or 1H-19F, while some can be applied to any nuclei of interest. For example, decoupling can be done through a specific spatial arrangement of the RF coil elements. In addition to this geometric decoupling, inductive decoupling and capacitive decoupling are other methodologies that are among the embodiments of the disclosure. Although inductive decoupling and capacitive decoupling apply to both active and passive decoupling, geometric decoupling is solely applicable for passive decoupling.

In some embodiments of the disclosure, passive decoupling is done in a frequency selective manner, while in other embodiments, the frequency selective passive decoupling is done using lumped element circuitry.

In analogous embodiments of the disclosure, active decoupling is done in a frequency selective manner, while in other analogous embodiments, the frequency selective active decoupling is done using lumped element circuitry, while in still other analogous embodiments, the active decoupling is done using direct current driven PIN diode circuitry.

When a substantial difference between the resonant frequencies of the nuclides exists, such as with 1H-13C, decoupling element design can be more straightforward. However, for instances where the resonant frequencies of the nuclides are quite close, such as 1H and 19F, which are separated by only ˜6% in frequency, decoupling is a very difficult problem when power intensive sequences are employed. Sufficient isolation between the RF pulse used for perturbation and that which is being observed upon relaxation must be maintained without decreasing the efficiency with which the MR signal from the targeted nuclide can be obtained. With the decoupling elements of the present disclosure, this is achieved by actively decoupling one of the coil elements, which can improve transmit and receive efficiency of the second radio frequency coil element. A passive decoupling element (tank circuit) used on a 19F coil element tuned to the 1H frequency has a high impedance at the operating frequency of the 19F coil element, leading to localized heating of the decoupling element. Therefore, structurally the decoupling element can be designed to operate under this condition during power intensive sequences. The high impedance of the decoupling element (at the 19F frequency) leads to high electric fields between the two ends of the decoupling element, and thus heating. The high electric fields can be reduced by increasing the distance between the two ends of the decoupling element, segmenting the junction with an additional capacitor, or larger footprint capacitor packages. Furthermore, the inductor wire of the decoupling element can be constructed of thicker gauge copper wire to reduce resistance.

E. Holding Assembly

The holding assembly is utilized primarily to contain the test sample and includes an at least partially enclosed space in which to place a test sample. This assembly may vary significantly in size, shape and configuration based upon the nature of the test sample and the configuration of the MR device or system. Nonetheless, certain characteristics of the holding assembly are constant as it cannot contain any magnetic or metal parts, yet should have a means for securing the test sample therein. As examples of such means, a simple restraining or locking mechanism may be employed for a chemical or material sample, while a “bite bar” or strap constraint may be used with a rodent or other animal species and a hand bar may be used for human or non-human primate subjects. It can be made from plastics, polymers, carbon fibres or other non-magnetic substances that can be rigidified to hold the weight of a test sample, yet still can be molded or shaped into appropriate sizes.

In some embodiments, the holding assembly fits at least partially into the detunable resonator, while in alternative embodiments, the holding assembly fits entirely into the resonator. When an RF coil element is utilized as the detunable resonator component, then as additional embodiments the holding assembly fits at least partially into the main magnet, while in alternative additional embodiments, the holding assembly fits entirely into the magnet.

F. Computing Device/Controller

For proper operation of the MR system and in order to utilize it for the methods of the disclosure, the magnet, as well as the gradient coil elements and cryogenic cooling unit if present, can be controlled by an integrated or, in preferred embodiments, external computing device, such as a computer, or a controller. Such computing device/controller exerts control over and maintains the homogeneity and stability of the magnetic field, which can be a critical element in obtaining reliable information from the MR system. It also may control the shimming of the magnetic field in response to perturbations caused by the local environment and the test sample.

In addition, the detunable resonator and the RF coil elements not only can be controlled in terms of their frequencies for transmittal and/or receipt of radiofrequencies, but also can record received RF signals to the computing device, which could be the same or different than the device employed for the magnet. These return signals can be processed and analyzed in order to provide the desired MR data from the system.

A computing device or controller is responsible for a number of functions, including, but not limited to, maintaining and controlling the homogeneity of the magnetic field, controlling the gradients sets permitting signal localization, tuning of the resonator to a MR detectable spin species, separately tuning each of the RF coil elements to a MR detectable spin species, modulating one spin species from the other spin species, controlling the drive circuitry connected to the resonator and that connected to the RF coil elements, generation of pulse sequences, as well as recording, processing and analyzing the MR signals produced by the spin species.

In cases where the signal is weak, this may also require the signal to be amplified, digitized, and filtered to extract the necessary information. In addition, the computing device or controller executes appropriate data processing steps, such as a Fourier transform or an image reconstruction, to convert the MR signals received into a format suitable for analysis by a skilled artisan, such as a MR spectrum from a MRS or a MR image from a MRI. This can include comparison of the signals from different pulses or pulse sequences, addition, subtraction, combination, or other modification of one or more results obtained from the MR signals. In certain embodiments, the computing device produces a MR spectrum from the MR signals, and in certain other embodiments, the computing device produces a MR image from the MR signals. In some embodiments, a single computing device or controller is responsible for these functions, while in other embodiments, more than one computing device or controller is responsible for these functions, while in still other embodiments, separate computing devices or controllers are responsible for each of these functions.

In certain embodiments, the computing device or controller is responsible for controlling the drive circuitry though issuing a set of instructions to perform at least one prescribed pulse sequence.

In preferred embodiments of the disclosure, the pulse sequence is selected from the group consisting of dynamic nuclear polarization (DNP), heteronuclear decoupling, difference nuclear Overhauser enhancement (DNOE), nuclear Overhauser effect spectroscopy (NOESY), rotating frame nuclear Overhauser effect spectroscopy (ROESY), distortionless enhancement by polarization transfer (DEPT), insensitive nuclei enhanced by polarization transfer (INEPT), chemical exchange saturation transfer (CEST), and magnetization transfer (MT).

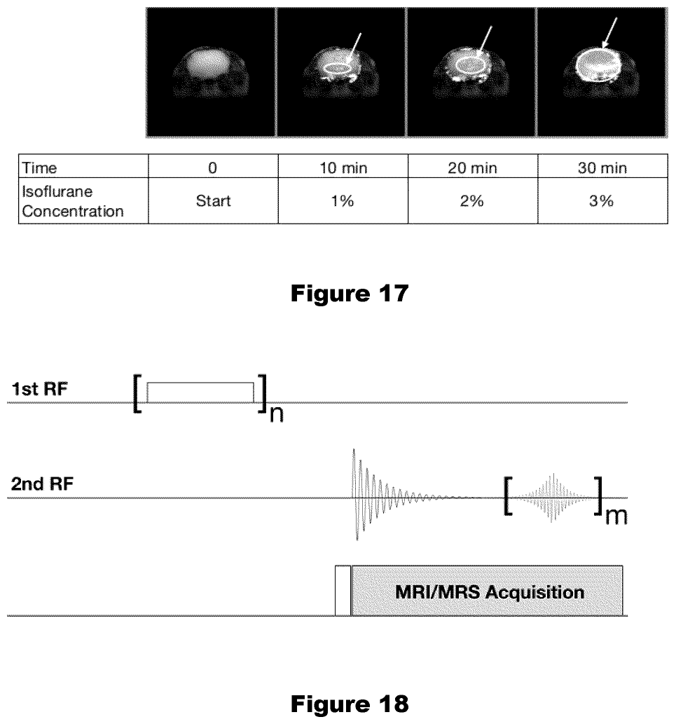

An example of a pulse sequence that can be used is provided in FIG. 18.

Examples of a computing device or controller suitable for use in the MR systems of the disclosure are: a computer workstation, a desktop computer, a laptop computer, a tablet computer, a handheld computer, an array of microprocessors connected in series, in parallel or other appropriate format within an instrument console or instrument control unit. For an MR system of the disclosure, one, or any combination, of these, and others, may be used as the computing device or controller as is required by the particular system components.

2. Methods of Use

Additional embodiments of the present disclosure provide methods of using the MR coil assemblies, holding assemblies, devices and systems of the disclosure. In a preferred embodiment, the method of using the MR coil assembly, holding assembly, device or system is for magnetic resonance spectroscopy (MRS). In another preferred embodiment, the method of using the MR coil assembly, holding assembly, device or system is for magnetic resonance imaging (MRI) instrument. Further, in additional embodiments, the methods are for therapeutic, diagnostic and research applications including, but not limited to, those described below.

MRS instruments are utilized for structural determinations of simple to complex organic and inorganic molecules and substances. The Larmor frequency is not constant among the observed nuclei in a compound or substance. Different observed nuclei of the observed nuclear species experience a slight variance or shift in their Larmor frequency based upon their binding partners, bond lengths, and bond angles. This shift occurs due to the nucleus being shielded from the B0 field by the effect of electrons or other factors interacting with a B0 field, which causes the individual nuclei to experience slightly different static magnetic fields. The frequency shift and the fundamental resonant frequency are directly proportional to the magnetic field strength; therefore, the ratio of the two values results in a field-independent, dimensionless value known as the chemical shift. The MR spectrum obtained has a frequency axis that corresponds to the chemical shift and an amplitude axis that corresponds to concentration. Along the frequency axis, specific nuclei give rise to a uniquely positioned single peak or multiple peaks. The area under the peak is directly related to the concentration of the specific nuclei. Information on the number of nuclei giving rise to a signal, the chemical shift of the signal, along with homonuclear and heteronuclear coupling patterns, are able to provide highly detailed structural information to those skilled in the art.

In some embodiments, the methods include one or more decoupling steps.

For example, the method further includes decoupling the second radio frequency coil element when the first radio frequency coil element transmits the first radio frequency signal.

For example, the method further includes decoupling the second transmitter coil element when the second radio frequency coil element receives the magnetic resonance signal.

For example, the method further includes decoupling the second radio frequency coil element when the first radio frequency coil element transmits and/or receives the magnetic resonance signal.

For example, when receiving signal from the first radio frequency coil, the device or system is programmed to decouple the second radio frequency coil and second transmitter coil (for example by sending 5V DC). In methods that include receiving signal from the second radiofrequency coil, the first radio frequency coil is passively decoupled and the device or system is programmed to actively decouple the second transmitter coil.

The methods can also include calibration steps where the first RF coil can be a transmitting and receiving coil. In such cases, the second radio frequency coil is decoupled, for example by sending a 5V DC, when transmitting and/or receiving on the first RF coil.

MRI instruments are employed for medical purposes, including diagnostic imaging of partial or full subjects, and to investigate the anatomy and physiology of a subject, as well as specific parts or regions of the subject's body. MRI is widely utilized in clinical and research settings to produce images of the inside of the human and animal bodies. As with MRS, MRI is based on detecting magnetic resonance (MR) signals from the nuclei of excited atoms upon the realignment or relaxation of the nuclear spin of atoms in an object being imaged (e.g., atoms in the tissue of a subject). Detected MR signals may be processed to produce images, which in the context of medical applications, allows for the investigation of internal structures and/or biological processes within the body, or any other sample, for diagnostic, therapeutic and/or research purposes. MRI provides an attractive imaging modality for biological imaging due to the ability to produce non-invasive images having relatively high resolution and contrast without the safety concerns of other modalities (e.g., without needing to expose the subject to ionizing radiation, e.g., x-rays, or introducing radioactive material to the body). Additionally, MRI is particularly well suited to provide soft tissue contrast, which can be exploited to image subject matter that other imaging modalities are incapable of satisfactorily imaging. Moreover, MR techniques are capable of capturing information about structures and/or biological processes that other modalities are incapable of acquiring.

In an embodiment of the disclosure, methods of imaging animals, including mammals, such as humans, live or not, whole or part thereof such as an organ or other region thereof.

In another embodiment of the disclosure, the MR coil assemblies, holding assemblies, devices and systems can be used in methods of imaging test samples such as cells, optionally 2D or 3D cell culture or tissues or synthetic or biosynthetic samples such 3D-printed tissues, organs, materials, and other samples, for example as produced by 3D-printing. For example, methods involving 2D or 3D cell cultures can be used to assess compounds comprising at least one isotope of the first spin species, optionally 13C, 15N, 19F or 31P, for their ability to for example, to penetrate or attach to cells, assess the metabolites produced and/or other properties of the compound.

A number of parameters have been found to be reasonably predictive of the ability of a compound to eventually be able to be developed as a pharmaceutical. These include the ability of an active compound to reach its site of intended action in the body of a treated subject. The MR coil assemblies, holding assemblies, devices and systems of the disclosure facilitate this analysis by being able to image the location of an active substance containing an MR-active nucleus and follow its appearance and disappearance over time. Hence, in other embodiments, the MR coil assemblies, holding assemblies, devices and systems can be in methods used for research on the adsorption, distribution, metabolic, and elimination fate of pharmaceutical, environmental or other test substances.

The following describes a particular example of this utility. MRI generally utilizes the hydrogen nuclear spins of the large amount of water molecules, each containing two hydrogen nuclei (i.e. protons), in most subjects (a human body, other organism, organ, material or tissue), although other nuclides have been used as well. It relies on detection of the protons of water molecules in order to form the images with the differences in various regions or tissues providing the necessary contrast. In attempting to detect specific drug molecules, however, this is not a viable approach, so attention can be directed towards other MR-active nuclei in the target compound, such as 13C, 15N, 19F or 31P. However, the natural abundances of 13C and 15N are low and phosphorous is not that common in drug molecules. In contrast, approximately 25% of approved drugs contain fluorine (Chem. Rev. 2014, 114(4), 2432-2506) with 19F being the only natural isotope, thus providing a viable target for MR studies. Unfortunately, 19F MRI is not a very sensitive modality, so fairly high concentrations typically need be present for accurate detection.

Nonetheless, 19F MRI remains very attractive as it provides quantitative images without ionizing radiation, does not have tissue depth limits, and lacks background signals. Although successfully used to study a number of biological processes, its utility for in vivo tracking of a drug remains a considerable challenge. Due to its low sensitivity, highest resolution images require quite significant local 19F concentrations (>80 mM) to generate high resolution images. Further, 19F signal splitting by adjacent nuclei and signal quenching by interaction with biomacromolecules has effectively excluded the possibility of directly imaging fluorinated drugs in vivo. As an example of the steps necessary to circumvent these limitations and permit the use of 19F MRI for investigating the PK-ADME (i.e. pharmacokinetics—absorption, distribution, metabolism, and excretion) properties of drugs, 19F MRI, a fluorinated liposomal drug delivery system prepared from fluorinated dendritic amphiphiles has been described that allowed the in vivo tracking of doxorubicin in tumor-carrying mice (Chem. Commun. 2018, 54, 3875-3878).

The MR assemblies, devices and systems of the disclosure facilitate the use of 19F MRI in vivo. Example 2 provides an illustration of this particular utility using a representative laboratory test animal being subjected to a representative treatment protocol of 19F to 1H have close resonance frequencies. Hence, extension to similar determinations for other MR-active nuclei is within the scope of this disclosure as well.

Subjects suitable to be assessed with the MR assemblies, devices and systems according to the present disclosure include, but are not limited to, mammalian and avian subjects. In addition, test samples comprising the detectable spin species described herein such as fluorine, can also be assessed. For example, the test sample can be cells in culture, a tissue sample or organ of a subject that has been administered (on contacted with) a compound comprising a detectable spin species such as a fluorinated compound. Alternatively, the test sample can be a 3D printed sample, such as a 3D cell culture or material containing cells, a 3D tissue like sample or organ, that for example has been contacted with (e.g. injected with, submerged in) the compound and/optionally subjected to a manipulation to assess one or more properties caused by the compound and/or manipulation. For example, the 3D tissue like sample may be injected with a compound to assess transport or localization. The test sample can also be a material or composition, comprising a detectable spin species in at least a portion thereof.

The test sample can also for example be any material including for example, a mud sample or other environmental sample for detecting explosive or other foreign material comprising a detectable spin species.

Mammals of the present disclosure include, but are not limited to, canines, felines, bovines, caprines, equines, ovines, porcines, rodents (e.g. rats and mice), lagomorphs, primates, humans, and the like, and mammals in utero. Any mammalian subject, including humans, is suitable. Human subjects of both genders and at any stage of development (i.e., neonate, infant, juvenile, adolescent, adult) can be studied according to the present disclosure. Illustrative avians according to the present disclosure include chickens, ducks, turkeys, geese, quail, pheasant, ratites (e.g., ostrich) and domesticated birds (e.g., parrots and canaries), and birds in ovo. The disclosure can also involve non-mammalian subjects, including reptiles and amphibians.

Particular embodiments of the disclosure are concerned with the imaging of mammalian subjects, such as mice, rats, dogs, guinea pigs, rabbits, cats, and pigs as well as humans, optionally for drug discovery and drug development purposes, livestock and horses for veterinary purposes, and humans for medical purposes, including the diagnosis and monitoring treatment for example of the conditions described herein.

Fluorine is used in many drugs and fluorine is used in a wide range of drug applications including anesthetics, antacids, anti-anxiety, antibiotics, anti-depressants, anti-fungal antibiotics, anti-histamines, antillipemics, anti-malarial, antimetabolites, appetite suppressants, arthritis/anti-inflammatory agents, psychotropic, steroids/anti-inflammatory agents as well as cannibinoids and psychedelic phenethylamines. The coil assemblies, holding assemblies, devices, systems and methods described herein can be used to detect localization of drugs comprising fluorine or a nuclide of a spin species described herein being developed to confirm drugs or their metabolites localize to the intended target and to monitor localization of existing drugs for example to monitor and/or optimize treatment regimens and doses. For example, the coil assemblies, holding assemblies, devices and methods described herein can be used to a assess if a brain acting drug is crossing the blood brain barrier, if a pancreas directed drug is entering the pancreas etc. In some embodiments, the amount of drug is quantitated. Such methods can be employed in drug development and for monitoring existing drugs.

The MR coil assemblies, holding assemblies, devices, and systems of the present disclosure can be used for the diagnosis of a range of medical conditions and guidance on the appropriate course of treatment, including, but not limited to, metabolic and/or endocrine disorders, gastrointestinal disorders, cardiovascular disorders, obesity and obesity-associated disorders, central nervous system disorders, bone and spine disorders, genetic disorders, hyperproliferative disorders, inflammatory disorders, immunity disorders and combinations thereof where the disorder may be the result of multiple underlying maladies. For example, disease detecting agents such as tumour antigen specific antibodies, that are labeled with a compound comprising a detectable spin species such as 19F, can be used to detect the presence of disease, and the extent of disease. For example in the case of administering antibody specific for a tumour antigen, the localization and size of the tumour may be determined. The methods described herein may also be used to monitor treatment response.

Further embodiments of the present disclosure will now be described with reference to the following examples. It should be appreciated that these examples are for the purposes of illustrating embodiments of the present disclosure, and do not limit the scope of the disclosure.

### EXAMPLES

### Example 1

A. Dual Channel Surface Coil Assembly

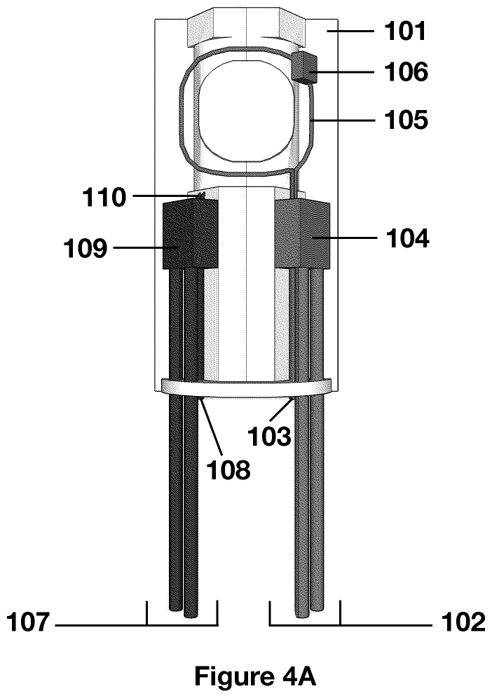

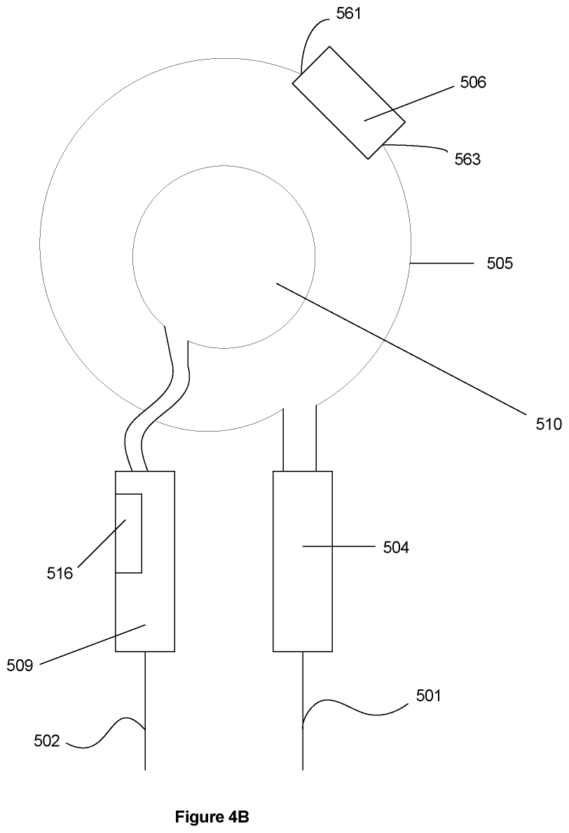

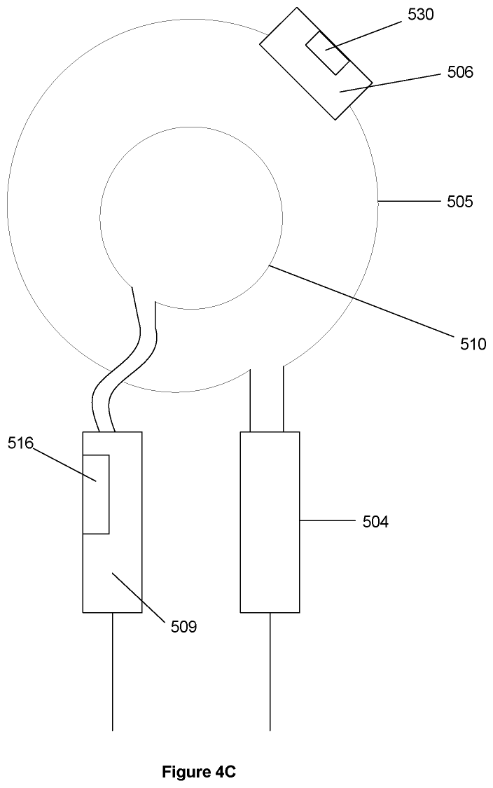

An example of a representative dual radio frequency (RF) coil system of a magnetic resonance (MR) device and/or system of the disclosure is assembled as shown in FIGS. 4 (top view), 5 (bottom view), 6 and 7.

The coil assembly includes a first radio frequency coil element (105) configured for transmitting a first radio frequency signal through a region of interest of a subject or test sample, said first radio frequency signal for exciting a first spin species in the region of interest. The coil assembly also includes a second radio frequency coil element (110) configured for resonating at a second radio frequency signal to receive a magnetic resonance signal from a second spin species from the region of interest.

In another embodiment, the second radio frequency coil element can further be configured to transmit a radio frequency signal through the region of interest, such that the radio frequency signal excites the second magnetic resonance detectable spin species in the region of interest. In this specific embodiment, the second radio frequency coil element can also be configured for resonating at the second radio frequency signal to receive the magnetic resonance signal from a second spin species from the region of interest. Thus, the second radio frequency coil element transmits and receives a signal. For example, the coil circuitry connected to the second radio frequency coil element can include a controller for activating the receive mode and/or transmit mode of the second radio frequency coil.

For example, the first spin species can be different from the second spin species. The first radio frequency signal and the second radio frequency signal can be separated by a frequency interval.

In other cases, the frequency interval is greater than zero. As an illustrative example: the first radio frequency signal is equal to 200 MHz; the second radio frequency signal is equal to 180 MHz; the frequency interval is equal to 20 MHz (e.g. |First radio frequency signal−Second radio frequency signal|=200 MHz−180 MHz=20 MHz). In this particular example, the frequency interval is equal to 10% of the first frequency and 11.11% of the second frequency

For example, the frequency interval can be less than 5% of the second frequency. For example, the frequency interval can be less than 10% of the second frequency. For example, the frequency interval can be less than 15% of the second frequency. The frequency interval can be less than 20% of the second frequency. The frequency interval can be less than 25% of the second frequency. The frequency interval can be less than 30% of the second frequency. The frequency interval can be less than 35% of the second frequency. The frequency interval can be less than 45% of the second frequency.

In some cases, the frequency interval can be equal to zero, such that the first and second radio frequency signals are the same. For example, the frequency interval can be zero and the first and second spin species can be the same when the coil assembly is used in traditional applications such as when protons are excited and detected.

The coil elements can be placed on a scaffold (101). The first and second radio frequency coil elements can be connected to the scaffold (101). The scaffold can include an internal surface and an external surface, and wherein the first radio frequency coil element is arranged on the external surface of the scaffold (101) and the second radio frequency coil element (110) is arranged on the internal surface of the scaffold (101).

For example, the scaffold can be part of the holding assembly of the MR device and/or system. Such scaffold can be 3D-printed using a variety of adequate plastics or other non-magnetic materials, although could also be made from carbon fiber, cardboard or another non-magnetic semi-rigid material that can be shaped or molded into the desired arrangement.

For example, attached to the scaffolding on the upper side is the RF coil element (105) for the X1-nucleus, along with a means to decouple this coil element (106). 105 is connected to its corresponding drive circuitry (104), as well as a means to match and/or tune the X1-nucleus channel (102), in this case, rods to mechanically adjust the coil electrical properties inside the magnet and thereby increase throughput and sensitivity. An additional connection (103) can be provided for the X1-channel to the balun and the imaging circuitry.

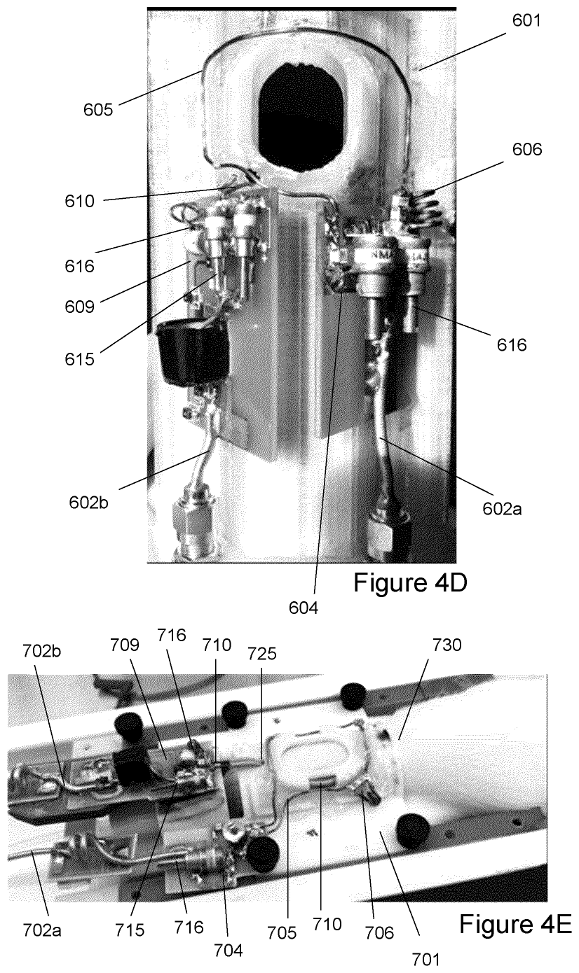

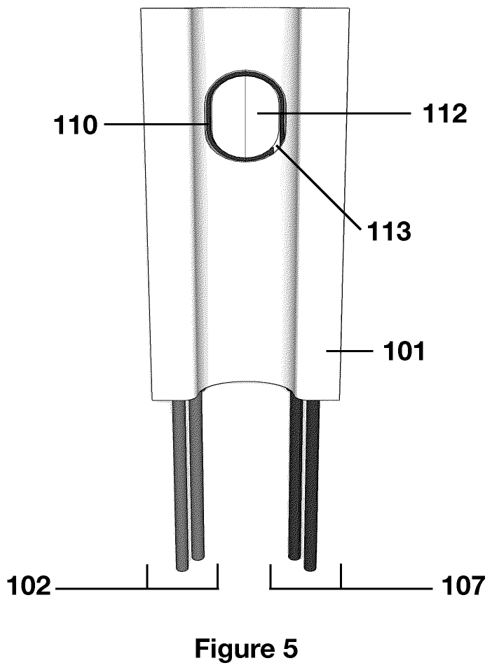

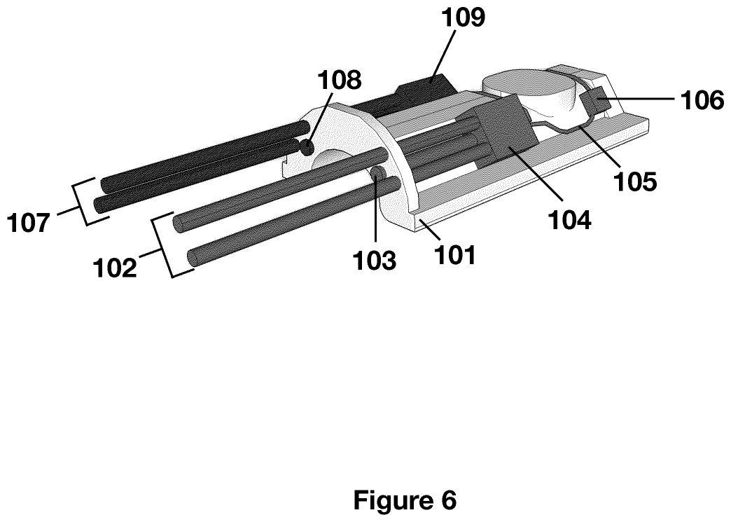

Similarly, in FIG. 4A are seen the X2-nucleus circuitry (109) and a connection for the X2-channel to the balun and the imaging circuitry (108). The RF coil element for the X2 nucleus (110) can be positioned on the opposite side of the scaffolding as shown in FIG. 5 and, as such, is closer to the test sample. In FIG. 5, element 113 is pointing to a groove that the RF coil element fits in. In this particular arrangement, 110 is just slightly smaller than 105 and circumnavigates the region of interest as defined by the positioning element 112. Indeed, a groove has been formed into the scaffolding to match with the size of 110 and secure it right around the test region. As can be seen better in the side view of FIG. 6 or the end view of FIGS. 7, 103 and 108 extend only to the end of the scaffolding, while the adjustment rods 102 and 107 extend significantly beyond the end of the scaffolding.

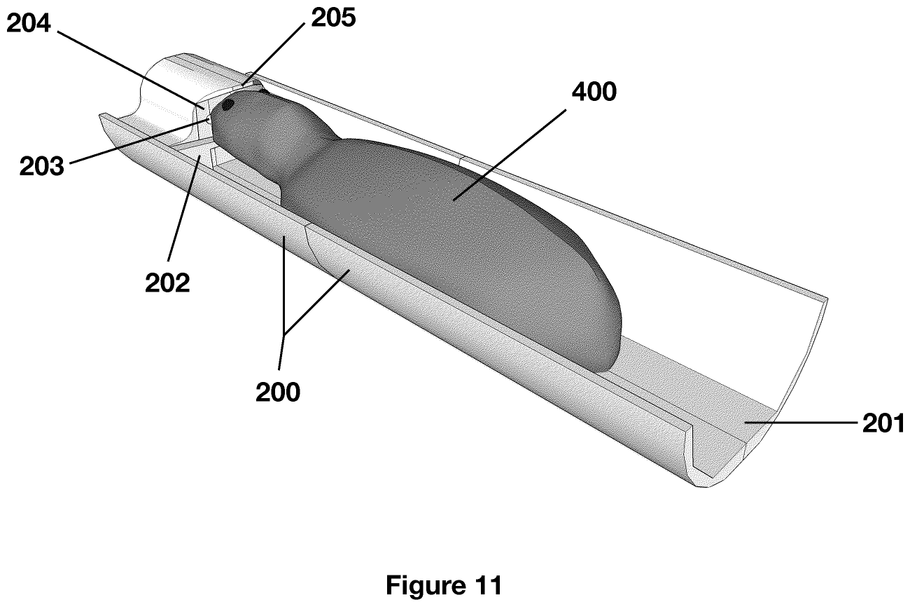

This scaffold can be part of the holding assembly, although it also could be a separate component. A cover (111) for the scaffolding can be used to cover the dual channel surface coil assembly (100), as illustrated in FIG. 12.

In another embodiment, the scaffold can include an additional transmitter coil. This additional transmitter coil can act as a resonator (i.e. in this specific case, a separate external resonator is no longer needed to transmit a radio frequency signal to the region of interest). This additional transmitter coil can be positioned on an internal surface or an external surface of the scaffold. For example, the additional transmitter coil and a first radio frequency coil element can be arranged on the external surface of the scaffold. Or, the additional transmitter coil and a second radio frequency coil element can be arranged on the internal surface of the scaffold.

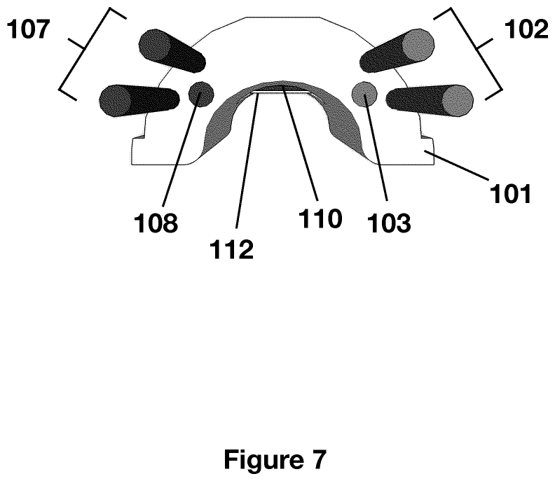

Referring to FIGS. 8A and 8B, there are shown decoupling circuits for the coil assembly of FIGS. 4 and 5. For example, these decoupling circuits can be active or passive.

For example, the first decoupling circuit can be configured to prevent coil coupling between the first radio frequency coil, the second radio frequency coil element and/or the transmitter coil element by disabling (e.g. turning off) the first radio frequency coil element when the second radio frequency coil element and/or the transmitter coil element is/are activated (e.g. turned ON).

For example, the first decoupling circuit can be configured to prevent coil coupling between the first radio frequency coil element and the second radio frequency coil element by disabling (e.g. turning off) the first radio frequency coil element when the second radio frequency coil element is activated (e.g. turned ON).

For example, the first decoupling circuit can be configured to prevent coil coupling between the first radio frequency coil element and the transmitter coil element by disabling (e.g. turning off) the first radio frequency coil element when the transmitter coil element is activated (e.g. turned ON).

For example, the first and second decoupling circuits can both be active decoupling circuits. In such configuration, the first and second decoupling circuits can each include a switch (e.g. a controllable switch, a semiconductor switch, a PIN diode, etc.). The switch can be activated (e.g. ON and OFF) by a bias current (e.g. 5 volts DC). For example, in operation, a bias current (e.g. 5 volt DC) can be applied to the switch of a decoupling circuit to detune the corresponding coil. When the bias current is received at the switch, the switch blocks current flow in the corresponding coil across the frequency span the switch is designed to operate, such the coil in question is isolated (e.g. turned OFF) from the rest of the coil assembly.

Referring to back FIG. 4A, a first decoupling circuit (106) can be configured to prevent coil coupling between: (1) the first radio frequency coil element and a transmitter coil element; and (2) the first radio frequency coil element and the second radio frequency coil element.

The transmitter coil element can optionally be external to the coil assembly. The first decoupling circuit can be configured to disable the first radio frequency coil element when the second radio frequency coil element is ON and/or when the transmitter coil element is ON.

In one embodiment, the first radio frequency decoupling circuit can be configured as a passive decoupling circuit. In another embodiment, the first decoupling circuit can be configured as an active decoupling circuit, such that the active decoupling circuit can be powered to decouple the first radio frequency coil element when the transmitter coil element is ON.

The decoupling circuit can have a junction at each end wherein each junction is connected to the first radio frequency coil element. The first decoupling circuit can be tuned to the second radio frequency signal. A separation distance between the junctions of the first decoupling circuit can be set to reduce the electric field caused by the proximity between the first and second radio frequency signals.

Referring to back FIG. 4A, the first decoupling circuit (106) is positioned on the first radio frequency coil element (105). For example, the transmitter coil element is external to the coil assembly. The transmitter coil element is generally found in a resonator, when such resonator is connected to the coil assembly. As such, the resonator includes the transmitter coil element for transmitting the second radio frequency signal for exciting the second spin species in the region of interest. Examples of such resonator is shown at 300 in FIGS. 12, 13, 14 and 15.

As shown in FIG. 4A, each junction of the first decoupling circuit (106) is connected to the first radio frequency coil element. The first decoupling circuit can be tuned to the second radio frequency signal. For example, the first decoupling circuit can be a passive decoupling circuit. The first decoupling circuit can include, among other elements, capacitors and inductors. Each of the values of the capacitors and inductors can be selected such that the decoupling circuit resonates at a desired frequency.

In some embodiments, the passive decoupling circuit consists of an inductor and capacitor pair placed in parallel, placed in series with the coil loop. Having a parallel inductor and capacitor has a resonant frequency given by f=1/root(LC), which is the frequency at which the capacitor/inductor pair is efficient at absorbing energy. This can be evaluated by bringing close a magnetic probe plugged into a network analyzer. If a nearby circuit is absorbing energy at a particular frequency, this magnetic probe will show a small dip in this S11 curve (reflection coefficient plot) on the network analyzer. The inductor can be adjusted till the dip in the S11 curve corresponds to the frequency of interest.

The first decoupling circuit can decouple the transmitter coil element from interacting with the first radio frequency coil element. For example, the first decoupling circuit (106) is configured to disable the first radio frequency coil element (105) when the transmitter coil element is active. A separation distance between the junctions reduces the electric field between the junctions caused by a high impedance between the junctions of the first decoupling circuit due to the proximity between the first and second radio frequency signals.

For example, a minimum separation distance can be calculated based on at least the voltage and electric field on the first radio frequency coil element (105). For example, a minimum separation distance can be determined based on the package size of fixed valued capacitors that are compatible to be used at the operated peak RF power. For example, an upper bound approach can be used to determine the separation distance between the junctions of the first decoupling circuit and the power value at the junctions. First, a peak RF power value can be determined. The peak RF power value will be used to drive the first radio frequency coil element (such as a 19F coil element). If the power value is say 1 kW, then the maximum voltage (V) induced in a coil (such as 50 ohm coil element) can approximated to be V=sqrt(1 kW*50)=223V. For example, if RF capacitors are used, then they can be rated for that voltage. For example, capacitors that can handle higher voltages proportionately are larger in dimensions to reduce the electric fields generated, where the Electric field (E) between the junctions is E=V/d, where V is voltage across the junction, and d is the separation distance of the junction.

For example, two capacitors (with voltage rating as determined above) can be used in series for the decoupling circuit. Having two capacitors divides the voltage and the electric fields across each capacitor by half, while the overall electric field between the passive decoupling circuit junction gets reduced compared to a single capacitor case since two series capacitors increases the junction separation to 2*d, as per the above E=V/d relation. For example, electric field induced heating in the inductor (due to resistive losses in copper wire), would be reduced.

For example, the first decoupling circuit (106) can include at least one capacitive element and an inductive element, which is in parallel with the capacitive element.

Returning to FIG. 8A, there is shown a second decoupling circuit (199) according to one embodiment. For example, the second decoupling circuit can be configured to prevent coil coupling between the second radio frequency coil element, the first radio frequency coil element and/or the transmitter coil element by disabling (e.g. turning off) the second radio frequency coil element when the first radio frequency coil element and/or the transmitter coil element is/are activated (e.g. turned ON).

The second decoupling circuit can be configured to decouple the second RF coil and the transmitter coil. In some embodiments, the decoupling circuit is configured to also decouple the second RF from the first RF coil as it was found that this could improve sensitivity of the first radio frequency coil. In such configurations, the second decoupling circuit prevents coupling between the second RF coil, the first RF coil and the second transmitter coil element.

For example, the second decoupling circuit can be configured to prevent coil coupling between the second radio frequency coil element and the first radio frequency coil element by disabling (e.g. turning off) the second radio frequency coil element when the first radio frequency coil element is activated (e.g. turned ON).

For example, the second decoupling circuit can be configured to prevent coil coupling between the second radio frequency coil element and the transmitter coil element by disabling (e.g. turning off) the second radio frequency coil element when the transmitter coil element is activated (e.g. turned ON).

The second decoupling circuit can be configured to prevent coil coupling between: (1) the second radio frequency coil element (110) and the transmitter coil element; and (2) the second radio frequency coil element and the first radio frequency coil element.

For example, the second decoupling circuit is connected to the second radio frequency coil element. The second decoupling circuit can be configured to disable the second radio frequency coil element (110) when the transmitter coil element operating at the second frequency is active and/or when the first radio frequency coil element is active.

For example, the second decoupling circuit includes a switch such as a controllable switch. The switch can be mechanical or electrical. The switch can be a PIN diode (such as PIN diode (D1) in FIG. 8A).

For example, the second decoupling circuit is configured to inhibit the second radio frequency coil element from resonating when the transmitter coil element transmits a signal.

For example, the second decoupling circuit can include a means for actively decoupling the second radio frequency coil element during a transmit phase of the transmitter coil element by applying a DC bias current which prevents the second radio frequency coil element from resonating at the resonant frequency of the second radio frequency coil element.

For example, there are power means for powering the second decoupling circuit.

For example, these power means can include power inputs from an RF cable and that is fed in with a Bias-T as illustrated in FIG. 8A, where the DC power signal is called the DC Bias.

As explained above, the specific diagrams in FIG. 8B can refer to a RF coil element acting as transceiver for a first magnetic resonance detectable spin species (X1 nucleus) with a passive decoupling circuit. FIG. 8A can refer to a RF coil element acting as a receiver for the second magnetic resonance detectable spin species (X2 nucleus) with an active decoupling circuit. Correspondence of the circuits to the hardware shown in FIGS. 4 and 5 are as indicated with 107, 108, 109 and 110 for the RF coil element for X2 and 102, 103, 104, 105 and 106 for the RF coil element for X1.

The pair of the first and second spin species can include one of: 19F and 1H; 31P and 7Li; 27Al and 13C; 6Li and 170; 10B and 15N; 6Li and 9Be; 9Be and 170; and 21Ne and 33S.

For example, the first magnetic resonance detectable spin species can be 19F and the second magnetic resonance detectable spin species can be 1H and vice versa.

The first and second spin species can be of the same isotope, for example 19F, wherein for example the coil assemblies and resonance systems are used to identify 19F containing compounds having different chemical shifts, for example when a 19F containing compound is in a membrane bound versus free state.

The first and second spin species can be in different molecular environments, independent of each other. Using 19F containing compounds as an example, the 19F compound can be bound and the 1H can be free (e.g. as present in bulk water) or bound (e.g: in the protein or membrane itself), or the 19F compound can be free and the 1H can be free or bound.

The coil assembly can include at least one tuner for separately tuning each of the first and second radio frequency coil elements to a magnetic resonance detectable spin species. For example, the first and second radio frequency coil elements can each have a tuning circuit to tune them to a desired frequency. For example, the tune circuit can include a voltage controlled capacitor (varactor) to enable remote tuning.

For example, the frequencies of the first and second coil elements can be changed to a functional range for the expected range of either nuclei (e.g. chemical shifts). For 1H, it represents about 3 kHz and for 19F about 42 kHz span at 7T. For example, changing nuclei may involve building new circuit boards with the same design but with different capacitor values to center it on the expected nucleus frequency (e.g. about 300 MHz for 1H and about 282 MHz for 19F at 7T).

The coil assembly can include a controller for controlling the circuitry (e.g. the drive circuitry). The coil assembly can also include means for powering and controlling the circuitry. For example, an On/Off switch can be located on the coil assembly to the turn it on or off. For example, coil assembly can be directly connected to a computing device, such that the computing device sends a pulse signal to the coil assembly for the MRI sequence. For example, the means for powering and controlling can be implemented by software on the computing device. The coil assembly can include a cover (111) for covering the scaffold (101) as shown in FIGS. 12 and 13.

Referring to FIG. 4B, shown is a coil assembly having a radio frequency coil 505 and a radio frequency coil 510 according to one example. The coil assembly of FIG. 4B includes elements similar to those shown in FIG. 4A. For example, coils 505 and 510 can be positioned on a scaffold to provide structural support to the coil assembly. The coil assembly is shown in FIG. 4B without being mounted on a scaffold for simplicity.

For example, the coils can be surface coils, birdcage coils, or any other suitable coils. The coils can include copper wire. The copper wire can have protective cover (e.g. plastic covering). For example, both coils of the coil assembly can have an arbitrary shape and can be arranged to provide geometric decoupling in the order of >10 dB. For example, the coil 505 can have a saddle shape for better coverage all around a target region (e.g. animal's head) when transmitting. For example, the coil 510 can have a smaller circular shape over the target region for better signal-to-noise ratio (SNR) for a region of interest.

The radio frequency coil 505 is connected to an electrical circuit 504. The coil 505 can be configured to transmit a first radio frequency signal to excite a first spin species in the region of interest. The coil 505 can be placed close (e.g. 2 cm) to the region of interest for example for better coverage and/or to reduce the amount of RF power used.

The radio frequency coil 510 is connected to an electrical circuit 509. The coil 510 can be configured to receive a second signal from a second spin species from the region of interest. The quality of the image produced by a processing device connected to the coil assembly is dependent, in part, on the strength of the signal received from the second spin species. For this reason, the receiving coil 510 can be placed in close proximity to a region of interest of a subject to improve signal reception strength. For example, the receiving coil can be placed within several millimeters of the subject's skin to image the subject's region of interest such as the brain (e.g. when the subject is a rodent, imaging up to 2-3 cm away from the receiving coil).

For example, the coil 510 can be as close as possible to the region of interest. For example, when mounted on a scaffold, coil 510 can touch the target region of the subject (e.g. head of a mouse). For example, protective cover (e.g. plastic cover, etc.) can be wrapped around the coil to protect it from being affected when touching the animal. Sources of losses in the receiver coil 510 can include: 1) thermal noise due to resistive components in the coil, 2) sample losses (these are losses in the subject (e.g. sample, animal, etc.) that are unavoidable and that primarily come from displacement currents in a conductive target region of the sample (e.g. brain, etc.)

The first radio frequency signal and the second radio frequency signal can be separated by a frequency interval. For example, the first radio frequency signal can be equal to 200 MHz and the second radio frequency signal can be equal to 180 MHz. In this example, the frequency interval is 20 MHz. Also, in this example, the frequency interval is equal to 10% of the first frequency and 11.11% of the second frequency. For example, the frequency interval can be less than 5% of the second frequency. For example, the frequency interval can be less than 10%, 15%, 20%, 25%, 30%, 35%, 40% or 45% of the second frequency.

The coil 505 is connected to an electrical circuit 504 and the coil 510 is connected to an electrical circuit 509. Each of electrical circuits 504 and 509 include wires or other conducting materials including capacitors, inductors, resistors of various types appropriate to drive the desired signals, current and power to the coils. Depending on the configuration, each of electrical circuits 504 and 509 can include variable capacitors, power supplies, pre-amplifiers, and/or other elements necessary to condition the desired signals, and supply current and power to the electrical circuits of the coils. For example, the coils 504 and 510 can each be connected to a tuning circuit to tune them to a desired frequency. For example, the purpose of the tuning circuit is to make the RF coil(s) sensitive to a particular frequency band, which is adjustable.

Transmission/sensing lines 501 and 502 are respectively connected to the electrical circuits 504 and 509 to transmit or sense signals to/from the coils 505 and 510 respectively. For example, line 501 and/or 502 and the detected signal separated from line 501 can be connected to a processing device (e.g. a computer, an MRI/MRS/NMR device, etc.).

A decoupling circuit 506 is connected to the coil 505. For example, the decoupling circuit 506 can be a passive decoupling circuit. For example, the passive decoupling circuit can be a tank circuit. For example, the tank circuit can include, among other elements, a combination of capacitors and inductors, such that each capacitor stores energy in the electric field between its plates, depending on the voltage across it, and each inductor stores energy in its magnetic field, depending on the current through it. For example, the decoupling circuit can absorb power at a particular frequency referred to as the resonant frequency.

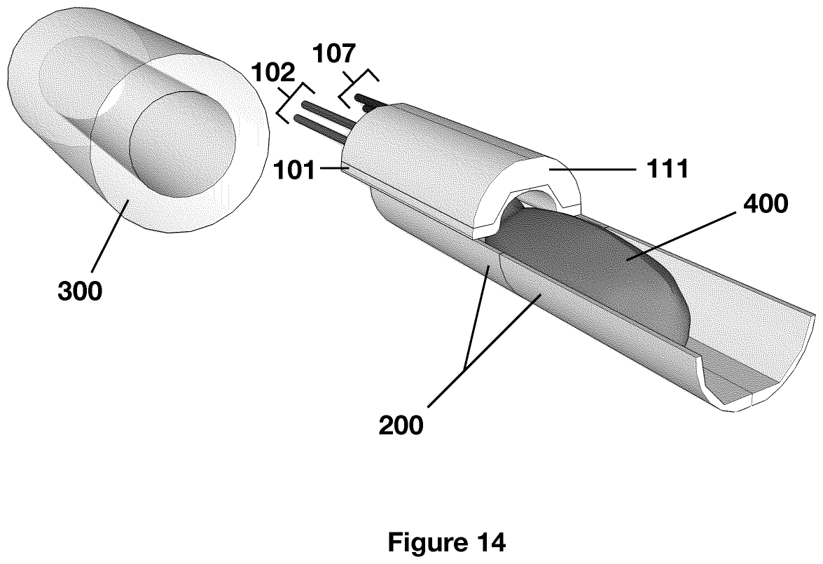

The decoupling circuit 506 can be tuned to a desired frequency, such as the resonant frequency. The purpose of the decoupling circuit 506 is to prevent coil coupling between: (1) between coils 505 and 510; and (2) between coil 505 and a transmitter coil when the coil assembly is used in combination with the transmitter coil (for e.g. the transmitter coil 300 as shown in the embodiment of FIG. 15). By decoupling coil 510, the decoupling circuit 506 prevents potential damages to the circuit 504 connected thereto from possibly induced voltages when the transmitter coil is active. For example, coupling between the coils may induce undesired RF energy on the target region of the subject, and reduce the effective RF field. For example, coil coupling may cause signal loss, and heating of the coil 505 if sufficient geometric decoupling is not present, which can damage to the coil 505, the circuit 504 and/or the coil assembly.

One of the advantages of having the passive decoupling circuit 506 is the elimination of the need for additional DC signal lines to control decoupling device of the coils (for active decoupling); thus, the simplification of the coil assembly design. As another advantage, because the decoupling circuit 506 is in series with the coil 505, the decoupling circuit 506 is always on and can allow near-instantaneous/faster sequence change, such that the coil 505 can acquire a signal in a matter of milliseconds (e.g. 0.1 ms-5 ms, etc.).

The decoupling circuit 506 has two junctions 561 and 563 connected to the coil 505. A separation distance between the junctions reduces the electric field between the junctions caused by a high impedance between the junctions of the first decoupling circuit due to the proximity between the first and second radio frequency signals. The minimum separation distance between the junctions can be calculated as described in the present subject matter.

For example, the coil assembly can be used in combination with an external transmitter coil (for e.g. the transmitter coil 300 as shown in the embodiment of FIG. 15). When such external transmitter coil or coil 510 are in transmit mode (e.g. transmit/receive at a target frequency respectively), the decoupling circuit 506 disables the coil 505 because having the decoupling circuit tuned to the target resonant frequency causes a high impedance (hence blocks current flow) at the target frequency.

A decoupling circuit 516 is located in the circuit 509. The purpose of the decoupling circuit 516 is to prevent coil coupling: (1) between coils 510 and 505; and (2) between coil 510 and a transmitter coil when the coil assembly is used combination with the transmitter coil (for e.g. the transmitter coil 300 as shown in the embodiment of FIG. 15). By preventing coil coupling, the decoupling circuit 516 protects the sensitive receiver equipment, including the coil 510 and the circuit 509. The decoupling circuit 516 decouples or detunes the receive coil 510 during the transmit RF phases of an imaging procedure using the coil assembly.

The decoupling circuit 516 can include a switch (e.g. a controllable switch, a semiconductor switch, a PIN diode, etc.). The switch can be activated (e.g. ON and OFF) by a bias current (e.g. 5 volts DC). The bias current can be fed to the decoupling circuit 516 through the transmission line 502, or fed in separately. When the bias current is fed through the transmission line, the transmission line represent a power means for powering the second decoupling circuit. For example, in operation, during a transmit phase of the transmit/receive cycle of an imaging operation (e.g. transmit phase by coil 505), a bias current (e.g. 5 volt DC) is applied to the switch of the decoupling circuit 516 to decouple or detune the coil 510. The bias current can be applied to the switch via the transmission line 502. When the bias current is received at the switch, the switch blocks current flow in the coil 510 across the frequency span the switch is designed to operate, such that coil 510 is isolated (e.g. turned OFF) from the rest of the coil assembly. The transmission line 502 is provided for both transferring received signals captured by coil 510 (when coupled) to a receiver (e.g. a receiving device, a processing device, etc.), and can be used to supply a bias current to the switch of the decoupling circuit when it is desirable to decouple the coil 510.

For example, suitable switches include ones that have a switching speed of less than or about 0.5 ms to less than or about 5 ms, for example less than 1 ms.

Referring to FIG. 4C, shown is a coil assembly according to one embodiment. The coil assembly of FIG. 4C includes elements similar to those in FIG. 4B and an adjustable tuning circuit 530 located at the decoupling circuit 506. The tuning circuit 530 can be used to tune the decoupling circuit 506 to a desired decoupling frequency. For example, the tune circuit 530 can include a voltage controlled capacitors (varactor), for remote tuning capabilities. FIGS. 4D and 4E show coil assemblies, according to other examples. Referring to FIG. 4D, the coil assembly includes a first radio frequency coil 605 and a second radio frequency coil 610. The coil assembly is mounted on a scaffold 601. The second radio frequency coil 610 is mounted on the inside (not shown) of the scaffold.

The first radio frequency coil 605 is connected to an electrical circuit 604. The second radio frequency coil 610 is connected to an electrical circuit 609. Each of electrical circuits 604 and 609 include wires or other conducting materials including capacitors, inductors, RF chokes, baluns, and PIN diodes, resistors of various types appropriate to drive the desired signals, current and power to the coil switching circuits. Depending on the configuration, each of electrical circuits 604 and 609 can include variable capacitors, power supplies, pre-amplifiers, and/or other elements necessary to drive the desired signals, current and power to the coils. For example, in each of the electrical circuits 604 and 609, in addition to the tuning capacitor, variable matching capacitors 615 and 616 can be included, which can be adjusted to impedance match the coil input to 50 Ohms, which is standard practice for RF devices interfaced in MRI.

For example, the coils 604 and 610 can each be connected to a tuning circuit to tune them individually to a desired frequency.

A passive decoupling circuit 606 is connected to the coil 605. The passive decoupling circuit 606 can be a tank circuit, including, among other elements, a combination of capacitors and inductors, such that each capacitor stores energy in the electric field between its plates, depending on the voltage across it, and each an inductor stores energy in its magnetic field, depending on the current through it. The passive decoupling circuit 606 is located in close proximity to the electrical circuit 604. For example, the passive decoupling circuit can be located anywhere on the coil 605.

An active decoupling circuit 616 is located in the circuit 609 for preventing coil coupling between coils 610 and 605, and between coil 610 and a transmitter coil when the coil assembly is used combination with the transmitter coil. The decoupling circuit 616 can include a switch (e.g. a controllable switch, a semiconductor switch, a PIN diode, etc.). The switch can be activated (e.g. ON and OFF) via a bias current (e.g. 5 volt DC/100 mA). The bias current signal can be fed to the decoupling circuit 616 through the line 602b. For example, in operation, during a transmit phase of the transmit/receive cycle of an imaging operation (e.g. transmit phase by coil 605), a bias current (e.g. 5 volt DC) is applied to the switch of the decoupling circuit 616 to detune the coil 610. The bias current can be applied to the switch via the line 602b.

Transmission/sensing lines 602a and 602b are respectively connected to the electrical circuits 604 and 609 to transmit or sense signals to/from the coils 605 and 610 respectively. Lines 602a and 602b can be connected to a processing device (e.g. a computer, an MRI/MRS/NMR device, etc.).

Referring to FIG. 4E, the coil assembly includes a first radio frequency coil 705 and a second radio frequency coil 710. The coil assembly is mounted on a scaffold 701. The second radio frequency coil 710 is mounted on the inside of the scaffold. The second radio frequency coil 710 extends from the electrical circuit 709 to the inside of the scaffold through an aperture 725 defined on the external surface of the scaffold 701. The scaffold 701 is mounted on the head of a rat 730. The first radio frequency coil 705 is connected to an electrical circuit 704. The second radio frequency coil 710 is connected to an electrical circuit 709. Each of electrical circuits 704 and 709 include wires or other conducting materials including capacitors, inductors, RF chokes, baluns, and PIN diodes, resistors of various types appropriate to drive the desired signals, current and power to the coils switching circuits. Depending on the configuration, each of electrical circuits 704 and 709 can include variable capacitors, power supplies, pre-amplifiers, and/or other elements necessary to drive the desired signals, current and power to the coils. For example, in each of the electrical circuits 704 and 709, in addition to the tuning capacitor, variable matching capacitors 715 and 716 can be included, which can be adjusted to impedance match the coil input to 50 Ohms, which is standard practice for RF devices interfaced in MRI.

A passive decoupling circuit 706 is connected in series to the coil 705. The passive decoupling circuit 706 can be a tank circuit. The passive decoupling circuit 706 can be positioned anywhere along the length of the coil 705. An active decoupling circuit 716 is located in the circuit 709 for preventing coil coupling between coils 710 and 705, and between coil 710 and a transmitter coil when the coil assembly is used combination with the transmitter coil. The decoupling circuit 716 can include a switch (e.g. a controllable switch, a semiconductor switch, a PIN diode, etc.). The switch can be activated (e.g. ON and OFF) by a bias current (e.g. 5 volt DC/100 mA). The power signal can be fed to the decoupling circuit 716 through the line 702b. For example, in operation, during a transmit phase of the transmit/receive cycle of an imaging operation (e.g. transmit phase by coil 705), a bias current (e.g. 5 volt DC) is applied to the switch of the decoupling circuit 716 to decouple or detune the coil 710. The bias current can be applied to the switch via the line 702b. Transmission/sensing lines 702a and 702b are respectively connected to the electrical circuits 704 and 709 to transmit or sense signals to/from the coils 705 and 710 respectively. Lines 702a and 702b can be connected to a processing device (e.g. a computer, an MRI/MRS/NMR device, etc.).

B. Holding Assembly

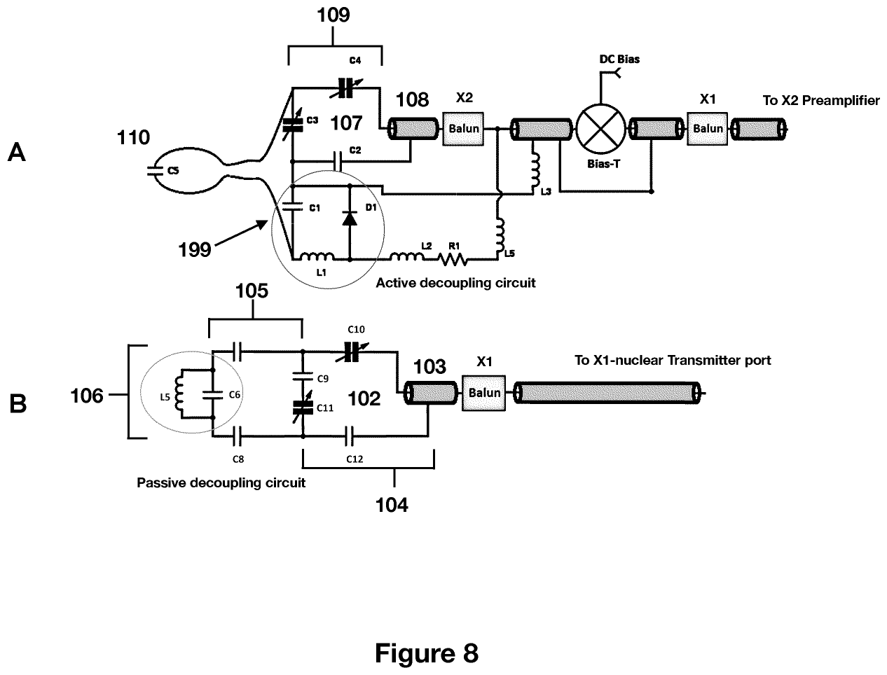

In addition to the portion containing the RF coil elements, the holding assembly can include other representative components illustrated in FIGS. 9 (top view) and 10 (side view) used for containing or positioning the subject or test sample.

For example, the holding assembly can include the coil assembly as described above and a holder (200) for placing the subject. For example, the holder comprises a partially enclosed space for placing the subject.

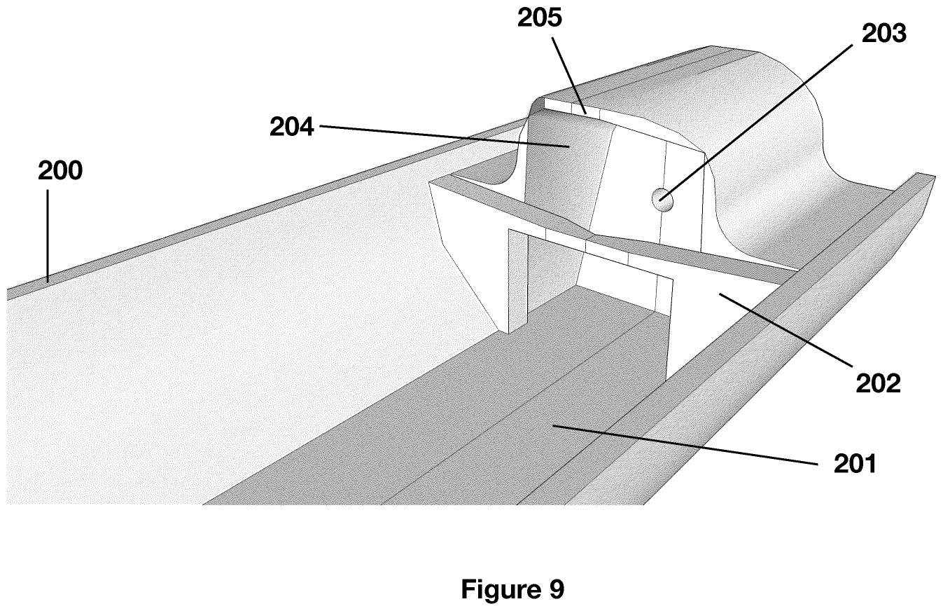

As shown in FIGS. 9, 10, 11, 12 and 13, the holder 200 can be a semi-cylindrical component containing a cavity (201) to hold the test sample, as well as a means for restraining the test sample (202). Such restraints can include straps or fasteners for chemical or materials samples, a bite bar for animal subjects, and a hand bar for human subjects. The composition of 200 can be the same as for the scaffold (101), although it could also be different. The intent is for the RF coil element scaffold (101) to fit securely into or at least match with a portion of the holding assembly as defined by 200. Another feature in this portion of the holding assembly is at least a connection port (203) for delivery of an external substance to the test sample or subject, such as but not limited to inert gas, anesthesia, odorants, or fluids.

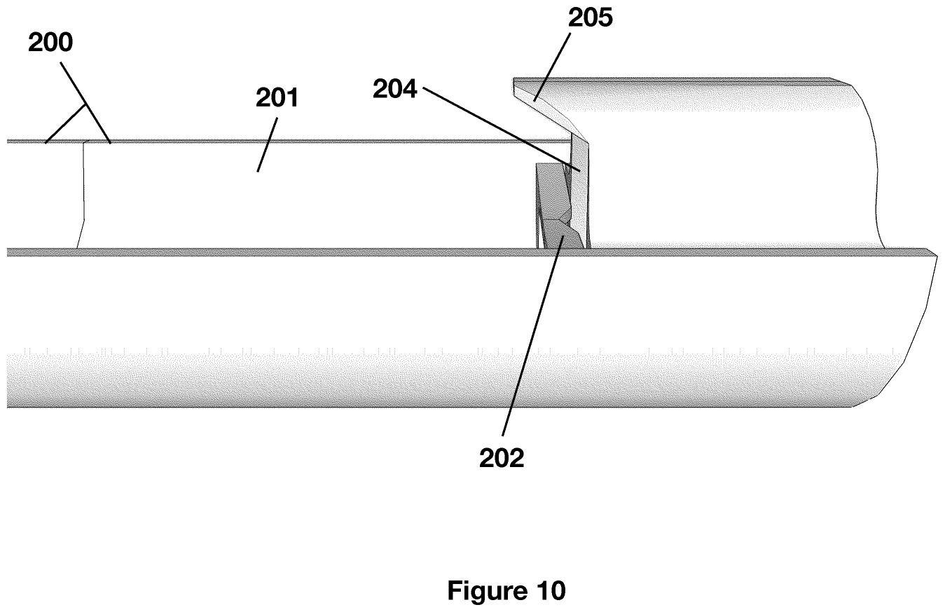

To partially enclose the test sample, the additional component 205 is employed, which creates a cavity (204) for delivery via 203 and/or to hold and protect the head of a subject. In the latter instance, this could also contain a nose cone for an animal subject. The enclosure created by 205 could also be longer to cover more or shorter to cover less of 200 than indicated in this example. In FIG. 11, the portion of the holding assembly just described with a subject (animal or human) as the test sample is shown.

In another embodiment, the holding assembly can include two or more coil assemblies positioned at various locations to cover multiple target regions of a subject. Each of the coil assemblies can have its own circuitry and/or decoupling circuits for performing the various functions as described above.

C. MR System

A system for magnetic resonance imaging (MRI) can include:

the holding assembly as described above and a resonator connected to the drive circuitry. The resonator includes the transmitter coil element for transmitting the second radio frequency signal for exciting the second spin species in the region of interest. For example, the resonator includes a cylindrical detunable resonator (300). A resonator tuner can be used for tuning the resonator to one magnetic resonance detectable spin species. The system can further include a magnet (500) and a magnet controller for controlling the homogeneity and stability of a magnetic field generated by the magnet. The magnet (500) can include a cylindrical opening for receiving the coil assembly and the resonator.

The system can also include a receiver unit that is connected to the drive circuitry for receiving the second radio frequency signals from the second radio frequency coil element. The system can further include an imager that reconstructs electronic image representations from the received second radio frequency signals.

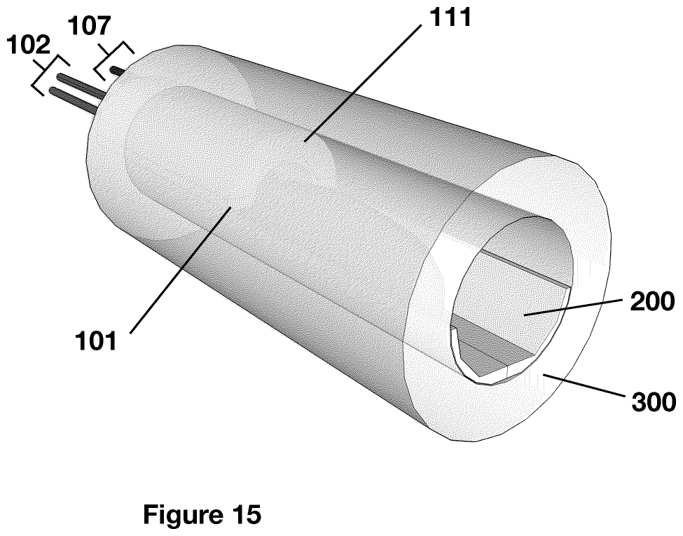

Referring to FIGS. 12-16, the dual channel surface coil assembly (100) and the holding assembly (200) as described above can be used in a MR system. For example, Coil assembly 100 is placed onto 200, then positioned over the upper portion of the test sample, for example the head of a subject, using the positioning rods 102 and 107 (FIG. 13). The top cover 111 is then secured over the dual channel coil assembly (FIG. 14), then placed into the cavity of the cylindrical detunable resonator (300). FIG. 15 shows the holding assembly 200 with the test sample completely inserted into 300. In order to obtain MR data, in the case of spectra for MRS, or an image for MRI, 300 is then inserted into the cylindrical opening in the main magnet (500) as illustrated in FIG. 16.

In use, a method of receiving magnetic resonance signals includes generating a magnetic field around a region of interest of a subject. The magnetic field can be generated using the main magnet. The method includes transmitting, with the first radio frequency coil element (105), a first radio frequency signal through the region of interest, said first radio frequency signal for exciting a first magnetic resonance detectable spin species. The method includes transmitting, with a second transmitter coil element (i.e. the resonator transmitter coil element), a second radio frequency signal through the region of interest, said second radio frequency signal for exciting a second magnetic resonance detectable spin species in the region of interest. The method includes capturing, with a second radio frequency coil element (110), a magnetic resonance signal from the second magnetic resonance detectable spin species; and processing the captured magnetic resonance signal.

For example, the first radio frequency signal and the second radio frequency signal can be separated by a frequency interval. For example, the first spin species is different from the second spin species. For example, the second magnetic resonance detectable spin species can be modulated by the first magnetic resonance detectable spin species;

The method further includes decoupling the second radio frequency coil element when the first radio frequency coil element transmits the first radio frequency signal. The method further includes decoupling the second transmitter coil element when the second radio frequency coil element receives the magnetic resonance signal. The method further includes decoupling the first radio frequency coil element (105) when the second radio frequency coil element receives the magnetic resonance signal.

For example, in operation, a coil assembly and a transmitter coil as described above can be connected to an MRI device and/or system. When transmitting signal with the first radio frequency coil, the MRI device or system can be programmed to detune the second radio frequency coil and the transmitter coil (e.g., by sending a 5V DC to both of them). In the case when receiving signal from the second radio frequency coil, the first radio frequency coil can be passively decoupled (e.g. by a decoupling circuit connected to the first radio frequency coil as described above), and the MRI can be programmed to actively decouple the transmitter coil (e.g., by sending a 5V DC to the transmitter coil).

For example, processing the captured magnetic resonance signal can include filtering and amplifying the captured magnetic resonance signal. The method further includes converting the processed magnetic resonance signal into a digital signal to obtain a magnetic resonance digital signal. For example, the method can include reconstructing and optionally displaying electronic image representations from the magnetic resonance digital signal. For example, the second transmitter coil element can be included within a resonator for transmitting the second radio frequency signal for exciting the second spin species in the region of interest.

### Example 2

A method for in vivo tracking of a compound in a subject optionally by tracking the compound in a tissue sample from the subject is provided herein. The subject can be a mammal. For example, the mammal can be a rat, a mouse, dog, guinea pig, rabbit, cat, pig, a livestock animal or horse. The mammal can be a human. The compound can be a drug for treating a disease. The compound can be a diagnostic agent. The method can be used for monitoring localization of the compound over a selected time interval.

The method can include introducing the subject or a test sample into a holding assembly or a device. The device can include devices as described in FIGS. 4, 5, 6, 7, 8, 9, 10, 11, 12, 13, 14, 15 and 16.

The subject has been or will be administered the compound. For example, the compound can be administered sometime after starting data acquisition for example to establish a baseline. Alternatively, it can be administered prior to starting the data acquisition.

Where the test sample is cells or a tissue, the cells or tissue can be contacted with (e.g. injected with, submerged in) the compound. In some embodiments, cells or tissue from a subject is assessed and the compound can be administered to the subject prior to removing the tissue or cells.

The method can also include receiving magnetic resonance signals as explained above, wherein the compound comprises at least one isotope of the first spin species, optionally 13C, 15N, 19F or 31P.

The method can include determining the position or positions of the compound or a metabolite thereof in the subject from the processed captured magnetic resonance signal.

The isotope can be 19F. The isotope can also be one of: 13C, 15N, 19F or 31P.

The coil assembly can be situated around a region of interest of the subject such as the head of the subject, or around a vial or other receptacle for holding the tissue sample. The coil assembly can also be adapted for conforming to any one of a number of different locations on the body of the subject. The coil assembly can be adapted for conforming around the subject's body or around an anatomical feature of interest such as the head, neck, chest, stomach, back, or a limb (such as arm, leg, etc.).

Localization/quantification of the spin species can be accomplished using a spin-echo or gradient-echo sequence preceded with a magnetization transfer (MT) pulse allowing magnetization from 19F to 1H, or vice versa.

For example, the method can includes producing an image, optionally wherein the level of compound is indicated by colour intensity in the image.

### Example 3

¹⁹F Magnetic Resonance Imaging In Vivo

A MR system is utilized for this representative imaging experiment. A dual channel coil assembly, as shown in FIGS. 12, 13, 14, 15 and 16, is employed with a 20 mm×30 mm loop RF coil element for 19F as the first magnetic resonance detectable spin species (X1) and a 20 mm loop RF coil element for 1H as the second magnetic resonance detectable spin species (X2).

As noted previously, the close resonance frequency of 19F to 1H (Table 1) complicates this situation, but provides the most difficult test for the MR systems of the disclosure. In considering potential approaches, direct irradiation or either nuclei, 1H and 19F require, too much time to be of utility. As an alternative strategy, difference NOE spectroscopy provides a solution, although few if any applications of DNOE in imaging have been reported. Here, indirect inverse irradiation focused on 19F is utilized to provide rapid and specific identification of the target molecule of interest while observing 1H, while circulating in a subject organism. This enables studying the adsorption of the fluorinated pharmaceutical in an organ or region of the body, determination of its distribution, and tracking of its metabolic fate of an active drug on a reasonable and useable timeframe.

A live Sprague-Dawley rat was secured in the bed of the representative holding assembly of Example 1B. The RF coil elements were situated such that the area around its head was encircled to provide complete coverage for the brain of the animal, then the assembly placed in the MRI system of Example 1C. Localization of the spin species of interest, 19F, was accomplished with a spin-echo (or, alternatively, gradient-echo) sequence preceded with a magnetization transfer (MT) pulse allowing magnetization from 19F to 1H. An example of a suitable pulse sequence is provided in FIG. 18. The rat was subjected to increasing stepwise amounts of isoflurane, an inhaled general anesthetic with chemical structure CF3-CHCl—O—CHF2, over 30 minutes (see Table 2), then the flow was halted.

TABLE 2
Anesthetic Treatment

| Time (min) | Isoflurane |
|---|---|
| 0 | start |
| 10 | 1% |
| 20 | 2% |
| 30 | 3% |
| 30.1 | off |

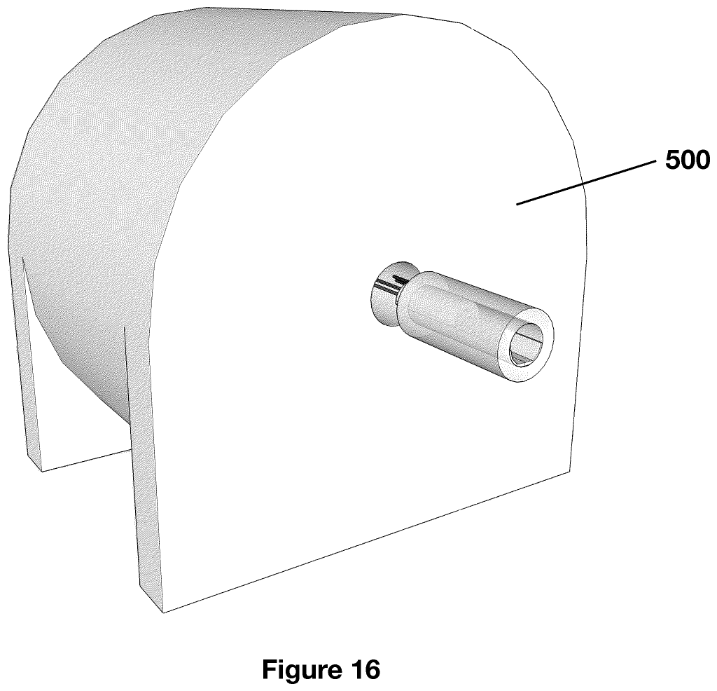

Anatomical MR images were obtained also using a standard spin-echo (or alternatively, gradient-echo) sequence. The MT pulse duration and strength were maintained constant prior to the signal acquisition by the surface coil. The increasing amount of 19F signal seen in the brain as imaged using the MR system is shown in FIG. 17, with the intensity of the signal as indicated by yellow/orange color (indicated in the Figure by an arrow pointing to the intensity of grey color circled in white, representing the level of the drug reaching the CNS in real time. Likewise, the disappearance of the concentration of isoflurane in the brain could be observed over the 30 minutes following cessation of anesthetic delivery (not shown).

The foregoing is illustrative of the present disclosure, and is not to be construed as limiting thereof.

1. A coil assembly, comprising:
a first radio frequency coil element configured for transmitting a first radio frequency signal through a region of interest of a subject or test sample, said first radio frequency signal for exciting a first spin species in the region of interest, and
a second radio frequency coil element configured for resonating at a second radio frequency signal to receive a magnetic resonance signal from a second spin species from the region of interest,
the first radio frequency signal and the second radio frequency signal being separated by a frequency interval;
corresponding circuitry connected to the first and second radio frequency coil elements;
a first decoupling circuit configured for preventing coil coupling between the first radio frequency coil, the second radio frequency coil element and/or a second transmitter coil element, the second transmitter coil element optionally being external to the coil assembly, the decoupling circuit comprising a junction at each end wherein
each junction is connected to the first radio frequency coil element,
the first decoupling circuit is tuned to the second radio frequency signal, and
a separation distance between the junctions of the first decoupling circuit is configured for reducing the electric field caused by the proximity between the first and second radio frequency signals; and
a second decoupling circuit configured for preventing coil coupling between the second radio frequency coil element, the first radio frequency coil element and/or the second transmitter coil element, the second transmitter coil element transmitting the second radio frequency signal for exciting the second spin species in the region of interest,
the second decoupling circuit being connected to the second radio frequency coil element,
the second decoupling circuit configured to disable the second radio frequency coil element when the second transmitter coil element operating at the second frequency is active and/or when the first radio frequency coil element is operating at the first radio frequency is active, and
a power means for powering the second decoupling circuit.

2. (canceled)

3. The coil assembly of claim 1, wherein the first decoupling circuit is configured for preventing coil coupling between the first radio frequency coil element and the second radio frequency coil element;
wherein the first decoupling circuit is configured for preventing coil coupling between the first radio frequency coil element and the transmitter coil element; or
wherein the first decoupling circuit is configured for preventing coil coupling between the first radio frequency coil element and the second radio frequency coil element and between the first radio frequency coil element and the transmitter coil element.

4. (canceled)

5. (canceled)

6. The coil assembly of claim 1, wherein the second decoupling circuit is configured for preventing coil coupling between the second radio frequency coil element and the first radio frequency coil element;
wherein the second decoupling circuit is configured for preventing coil coupling between the second radio frequency coil element and the transmitter coil element; or
wherein the second decoupling circuit is configured for preventing coil coupling between the second radio frequency coil element and the first radio frequency coil element and between the second radio frequency coil element and the transmitter coil element.

7. (canceled)

8. (canceled)

9. (canceled)

10. The coil assembly of claim 1, wherein the first decoupling circuit comprises:
at least one capacitive element; and/or
an inductive element which is in parallel with the capacitive element.

11. The coil assembly of claim 1, wherein the second decoupling circuit is configured to inhibit the second radio frequency coil element from resonating when the transmitter coil element transmits a signal.

12. (canceled)

13. The coil assembly of claim 1, further comprising a scaffold, wherein the first and second radio frequency coil elements are connected to the scaffold, and
optionally wherein the first radio frequency coil element is arranged on an external surface of the scaffold and the second radio frequency coil element is arranged on an internal surface of the scaffold.

14. (canceled)

15. The coil assembly of claim 1, wherein the frequency interval is less than 35% of the second frequency;
wherein the frequency interval is less than 30% of the second frequency;
wherein the frequency interval is less than 25% of the second frequency;
wherein the frequency interval is less than 20% of the second frequency;
wherein the frequency interval is less than 15% of the second frequency; or
wherein the frequency interval is less than 10% of the second frequency.

16. (canceled)

17. (canceled)

18. (canceled)

19. (canceled)

20. (canceled)

21. The coil assembly of claim 1, wherein a pair of the first and second spin species comprises one of:
¹⁹F and ¹H;
³¹P and ⁷Li;
²⁷Al and ¹³C;
⁶Li and ¹⁷O;
¹⁰B and ¹⁵N;
⁶Li and ⁹Be;
⁹Be and ¹⁷O; and
²¹Ne and ³³S.

22. The coil assembly of claim 1, further comprising at least one tuner for separately tuning each of the first and second radio frequency coil elements to a magnetic resonance detectable spin species; and
a drive circuitry controller.

23. (canceled)

24. (canceled)

25. A holding assembly, comprising:
one or more coil assemblies as claimed in claim 1; and
a holder for placing the subject.

26. (canceled)

27. A device for magnetic resonance imaging (MRI), comprising the holding assembly as claimed in claim 25; and
a resonator connected to a drive circuitry, the resonator comprising the transmitter coil element configured for transmitting the second radio frequency signal for exciting the second spin species in the region of interest.

28. The device of claim 27, wherein the resonator comprises a cylindrical detunable resonator; and
wherein the device further comprises a resonator tuner for tuning the resonator to one magnetic resonance detectable spin species.

29. (canceled)

30. A system comprising the device of claim 27, further comprising a magnet and a magnet controller for controlling the homogeneity and stability of a magnetic field generated by the magnet.

31. The system of claim 30 wherein the magnet comprises an opening for receiving the coil assembly and the resonator; and
wherein the system further comprises a receiver unit connected to the drive circuitry for receiving the second radio frequency signals from the second radio frequency coil element and
an imager that reconstructs electronic image representations from the received second radio frequency signals.

32. (canceled)

33. (canceled)

34. A method of receiving magnetic resonance signals, comprising:
generating a magnetic field around a region of interest of a subject or a test sample;
transmitting, with a first radio frequency coil element, a first radio frequency signal through the region of interest, said first radio frequency signal for exciting a first magnetic resonance detectable spin species in the region of interest;
transmitting, with a second transmitter coil element, a second radio frequency signal through the region of interest, said second radio frequency signal for exciting a second magnetic resonance detectable spin species in the region of interest, wherein
the first radio frequency signal and the second radio frequency signal are separated by a frequency interval,
the second magnetic resonance detectable spin species is modulated by the first magnetic resonance detectable spin species;
capturing, with a second radio frequency coil element, a magnetic resonance signal from the second magnetic resonance detectable spin species; and
processing the captured magnetic resonance signal.

35. (canceled)

36. (canceled)

37. The method of claim 34 further comprising decoupling the second radio frequency coil element when the first radio frequency coil element transmits the first radio frequency signal;
decoupling the second transmitter coil element when the second radio frequency coil element receives the magnetic resonance signal; or
decoupling the first radio frequency coil element when the second radio frequency coil element receives the magnetic resonance signal.

38. (canceled)

39. (canceled)

40. (canceled)

41. (canceled)

42. (canceled)

43. (canceled)

44. (canceled)

45. (canceled)

46. The method of claim 34, wherein a pair of the first and second spin species comprises one of:
¹⁹F and ¹H;
³¹P and ⁷Li;
²⁷Al and ¹³C;
⁶Li and ¹⁷O;
¹⁰B and ¹⁵N;
⁶Li and ⁹Be;
⁹Be and ¹⁷O; and
²¹Ne and ³³S.

47. (canceled)

48. (canceled)

49. (canceled)

50. (canceled)

51. A method for tracking of a compound in a subject or test sample, the method comprising:
a. introducing the subject or test sample into a holding assembly, device or system, wherein the subject has or will be been administered the compound and/or the test sample has been or will be contacted with (e.g. injected with, submerged in) the compound;
b. receiving magnetic resonance signals according to the method of any one of claims 34-51, wherein the compound comprises at least one isotope of the first spin species, optionally ¹³C, ¹⁵N, ¹⁹F or ³¹P;
c. optionally processing the captured magnetic resonance signal to obtain an image; and
d. determining the position or positions of the compound or a metabolite thereof in the subject or test sample from the processed captured magnetic resonance signal.

52. The method of claim 51, wherein the at least one isotope is ¹⁹F.

53. (canceled)

54. (canceled)

55. (canceled)

56. The method of claim 51, wherein localization of the spin species is accomplished using a spin-echo or gradient-echo sequence preceded with a magnetization transfer (MT) pulse allowing magnetization transfer from ¹⁹F to ¹H or vice versa.

57. The method of claim 51, wherein the method further comprises producing an image, and optionally wherein the level of compound is indicated by colour signal intensity in the image.

58. (canceled)

59. (canceled)

60. (canceled)

61. The method of claim 51, wherein the compound is a drug for treating a disease; or
wherein the compound is a diagnostic agent.

62. (canceled)

63. (canceled)

64. The method of claim 51 wherein the test sample is a tissue and/or comprises cells, for example a 2D or 3D cell culture, optionally a 3D printed tissue like structure or organ; or
wherein the region of interest is selected from brain, lungs, spines, intestines, muscle, or liver.

65. (canceled)

66. (canceled)
